(19) United States
(12) Patent Application Publication
(10) Pub. No.: US 2025/0101627 A1
(43) Pub. Date: Mar. 27, 2025

(54) PREPARATION METHOD OF COLLAGEN MATERIAL STRIPPED FROM ELECTRODE AND USE OF COLLAGEN MATERIAL

(71) Applicant: EAST CHINA UNIVERSITY OF SCIENCE AND TECHNOLOGY, Shanghai (CN)

(72) Inventors: Xue QU, Shanghai (CN); Miao LEI, Shanghai (CN); Changsheng LIU, Shanghai (CN)

(73) Assignee: EAST CHINA UNIVERSITY OF SCIENCE AND TECHNOLOGY, Shanghai (CN)

(21) Appl. No.: 18/832,129

(22) PCT Filed: Nov. 11, 2022
(86) PCT No.: PCT/CN2022/131287

§ 371 Date: Jul. 23, 2024

(30) Foreign Application Priority Data
Jan. 27, 2022 (CN) 202210102037.X
Jan. 27, 2022 (CN) 202210102038.4
Jan. 27, 2022 (CN) 202210102039.9
Jan. 27, 2022 (CN) 202210102043.5

(51) Int. Cl. C25D 9/02; A61L 27/24; A61L 27/52; C25B 3/09; C25B 15/02; C25D 13/06; C25D 17/10

(52) U.S. Cl. CPC C25D 9/02; A61L 27/24; A61L 27/52; C25B 3/09; C25B 15/02; C25D 13/06; C25D 17/10

(57) ABSTRACT

A preparation method of a collagen material stripped from an electrode and a use of the collagen material are provided. The preparation method of a collagen material stripped from an electrode includes the following step: subjecting a collagen solution including hydrogen peroxide and/or acetic acid to electro-deposition (EDP) to obtain the collagen material on the electrode. The preparation method can lead to a collagen material directly on a surface of an electrode.

### CROSS REFERENCE TO THE RELATED APPLICATIONS

This application is the national phase entry of International Application No. PCT/CN2022/131287, filed on Nov. 11, 2022, which is based upon and claims priority to Chinese Patent Applications No. 202210102037.X, filed on Jan. 27, 2022, No. 202210102039.9, filed on Jan. 27, 2022, No. 202210102043.5, filed on Jan. 27, 2022, and No. 202210102038.4, filed on Jan. 27, 2022, the entire contents of which are incorporated herein by reference.

### TECHNICAL FIELD

The present application relates to a preparation method of a collagen material stripped from an electrode and a use of the collagen material, and belongs to the field of biological macromolecular assembly.

### BACKGROUND

Collagen is one of the most abundant proteins in vertebrates. Because collagen has low immunogenicity and high biocompatibility and can promote cell proliferation and wound healing, collagen has been widely used in various biomedical materials. Collagen has triple helix structures. Under the guidance of some endogenous signals in vivo, the triple helix structures can be assembled hierarchically and orderly. That is, the triple helix structures can be hierarchically assembled into collagen microfibers, collagen fibrils, and collagen fibers to finally produce tissue structures.

In vitro, a solution casting method is often used to process simple collagen into a collagen biomaterial. The solution casting method includes the following steps: adjusting a collagen solution to a pH of 7.0, then placing in a mold, and incubating at 37° C. for a specified period of time to complete the hierarchical assembly of collagen. However, the solution casting method has the following disadvantages: 1. It is not easy to prepare various heteromorphic collagen materials. 2. It takes several hours or even a night to prepare a collagen material. 3. A prepared collagen material has collagen fibers disorderly arranged inside, a low collagen arrangement density, and opaque appearance.

The electro-deposition (EDP) technology is a relatively-advanced method to prepare a collagen material. A principle of the EDP technology is as follows: An electric field is applied to an acidic solution of collagen to drive the electrophoretic migration of collagen molecules to a cathode region. Because an electrochemical reaction (usually a water electrolysis reaction) at a cathode can increase a pH of a solution near the electrode, collagen swimming to a collagen isoelectric point region will be precipitated to produce a collagen material. Compared with the solution casting method, the EDP technology has the following advantages: 1. The EDP process is generally relatively fast, and can be completed within about 30 min to 60 min. 2. The EDP technology allows the shaping of a material. 3. A prepared collagen material has a microstructure with orientation characteristics, dense collagen arrangement, and transparent appearance. The EDP technology also has obvious disadvantages: Because an isoelectric point region is usually located at a specified position in an electrolyte, a collagen material cannot be deposited directly on an electrode, which is not convenient to material acquisition and is also not convenient to shaping of a collagen material based on a shape of an electrode (such as preparation of a collagen material with a tubular shape or an irregular shape). In addition, a distance between two electrodes is very small and is generally 1 mm to 2 mm, which is inconvenient for operations (An electrochemical fabrication process for the assembly of anisotropically oriented collagen bundles, Biomaterials 29 (2008) 3278-3288; and Tenogenic Induction of Human MSCs by Anisotropically Aligned Collagen Biotextiles, Adv. Funct. Mater. 2014, 24, 5762-5770).

In addition, some researchers have used a pulsed EDP technology to acquire a collagen material on an electrode. A key strategy of the pulsed EDP technology is as follows: 50% ethanol (an organic solvent) is added to an electrolyte (which may be intended to reduce the solubility of collagen), and a pulse voltage is applied (which is intended to reduce hydrogen bubbles generated on a cathode to facilitate the deposition of collagen). The pulsed EDP technology has the following disadvantages: The organic solvent may cause a structural change of collagen. The uniformity of a material structure produced at an electrode is not easy to control. It takes as long as 1.5 hours to complete the pulsed EDP technology. The pulsed EDP technology can only lead to an opaque material (Fabrication of free standing collagen films by pulsed electrophoretic deposition, Biofabrication 11, 2019, 045017).

Therefore, it is necessary to develop a novel preparation method to overcome the above shortcomings, so as to acquire a desired collagen material rapidly and effectively.

### SUMMARY

According to a first aspect of the present application, a preparation method of a collagen material stripped from a cathode is provided. In the preparation method, because acetic acid is a weak acid, a specified buffering capacity can be provided to make a pH near a cathode close to an isoelectric point of collagen, such that a collagen material can be obtained directly on a surface of the cathode. A shape of the cathode can be changed to facilitate the preparation of collagen materials of various heteromorphic structures. A cathode reaction is a decomposition reaction of hydrogen peroxide, and does not produce bubbles, which is conducive to acquisition of a collagen material with very uniform and dense appearance, and can also solve the problem that a prepared collagen film may have a thin upper part and a thick lower part due to a gravity especially when horizontal electrodes are adopted. A distance between two electrodes can be controlled at a centimeter grade, which is convenient to operations. Only a short preparation time is required. The collagen material has the following characteristics: The collagen material is composed of short range-oriented amorphous collagen microfibers, and has dense collagen arrangement, transparent appearance, and a uniform structure, which is conducive to acquisition of excellent mechanical and optical properties. The collagen material has a plastic deformation ability. Collagen microfibers in the collagen material are linked through non-covalent bonds, and thus the collagen material can be re-dissolved in a solvent and can be recycled by an EDP technology, that is, recycling of the collagen material is allowed.

The preparation method of a collagen material stripped from an electrode includes the following step:
subjecting a collagen solution including hydrogen peroxide and/or acetic acid to EDP to obtain the collagen material on the electrode.

Optionally, a pH of the collagen solution is 1.5 to 4.0.

Optionally, a concentration of the collagen solution is 1 mg/mL to 20 mg/mL.

Optionally, a volume percentage of the hydrogen peroxide in the collagen solution is 5% to 17%.

Optionally, the EDP is conducted under the following conditions:

temperature: 0° C. to 30° C.; and time: 8 min to 60 min.

Optionally, during the EDP, a current density is 0.5 mA/cm² to 10 mA/cm² and a voltage is 0.22 V/cm² to 1.67 V/cm².

Optionally, during the EDP, a distance between two electrodes is 1.0 cm to 2.5 cm.

Optionally, a cathode is one selected from a stainless steel, a carbon paper, a carbon cloth, a Pt electrode, a gold electrode, a graphite electrode, and a Ti electrode.

Optionally, an anode is one selected from a stainless steel, a carbon paper, a carbon cloth, a Pt electrode, a gold electrode, and a graphite electrode. An anode material is not Ti.

Optionally, the preparation method includes the following steps:
S1. mixing a collagen solution-containing and the acetic acid to obtain a first collagen solution;
S2. adding a hydrogen peroxide-containing mixture to the first collagen solution to obtain a second collagen solution; and
S3. placing the second collagen solution in an electrolytic cell, and conducting the EDP to obtain the collagen material.

According to an embodiment of the present application, the preparation method includes the following steps:
S1. preparation of a collagen solution: adding acetic acid to a collagen raw material to completely dissolve the collagen raw material, adjusting a pH of a final solution to 1.5 to 4.0, and concentrating a resulting system to obtain a first collagen solution with a concentration of 1 mg/mL to 20 mg/mL;
S2. adding a hydrogen peroxide standard solution to the first collagen solution obtained in S1 to obtain a second collagen solution with a final hydrogen peroxide volume percentage of 5% to 17%, stirring the second collagen solution to remove bubbles, and placing the second collagen solution at 0° C. to 10° C. for later use;
S3. arranging a titanium sheet as a cathode and platinum as an anode in parallel in an electrolytic cell, and slowly adding the second collagen solution obtained in S2 to the electrolytic cell, where a distance between the two electrodes is controlled at 0.5 cm to 3.0 cm; and
S4. conducting EDP at a constant voltage or a constant current for 10 min to 60 min to obtain a collagen gel film that can be stripped from the cathode.

In the preparation method of the present application, the collagen material can be obtained directly on the electrode. The distance between the two electrodes is increased from a millimeter scale to a centimeter scale, and is significantly larger than a distance of 2 mm between two electrodes that is commonly adopted, which greatly facilitates the arrangement and subsequent operations of the electrodes. For example, the distance between the two electrodes can be controlled at 0.5 cm to 3.0 cm and preferably at 1.0 cm to 1.5 cm.

Optionally, an arrangement manner of the two electrodes includes: vertically arranging the two electrodes in parallel in an electrolytic cell, or horizontally arranging the two electrodes in parallel in the electrolytic cell. When the electrodes are arranged either horizontally or vertically, a collagen material can be prepared. However, it is found that a material prepared with the electrodes arranged vertically easily has a thin upper part and a thick lower part due to a gravity, which can be avoided when the electrodes are arranged horizontally.

Optionally, in S1, a mass of the collagen raw material is adjusted to make a final collagen solution have a concentration of 5 mg/mL to 10 mg/mL. In the present application, through repeated tests, an organic solvent is not required in an electrolyte, and other parameters in the preparation method can be adjusted to allow the preparation of free-standing collagen (gel film) (that is, the collagen can be stripped from an electrode and become an independent material) while effectively preventing the generation of bubbles at a cathode. The mass of the collagen raw material added can be changed to adjust the concentration of the final collagen solution. When the concentration of the final collagen solution is higher than 20 mg/mL, the solution loses fluidity; and excellent results can be obtained usually when the concentration of the final collagen solution is in a range of 1 mg/ml to 20 mg/mL.

In the present application, a variety of commercially-available collagen products may be adopted as the collagen raw material. For example, the collagen raw material used in the embodiment of the present application comes from Shanghai Haohai Biological Technology Co. Ltd. The free-standing means that another substrate is not adopted as a support.

Optionally, in S1, a final concentration of acetic acid in a dialysis solution needs to be determined based on a pH of a collagen electrolyte. If the concentration of acetic acid in the dialysis solution is too low, an amount of acetic acid in the collagen electrolyte in a dialysis bag will be reduced, which reduces the fluidity of collagen molecules during electrophoresis in a subsequent electrochemical reaction. If the concentration of acetic acid in the dialysis solution is too high, an amount of acetic acid in the collagen electrolyte will be increased, which easily makes collagen molecules fail to be deposited on a surface of the cathode in a subsequent electrochemical reaction. Actual operations are as follows: the collagen raw material is added to ultrapure water (UPW), a resulting mixture is stirred, and acetic acid is added dropwise, during which a pH of a resulting system is monitored by a pH meter until the pH is in a pH range specified in the present application to completely dissolve the collagen raw material.

Optionally, in S2, the final hydrogen peroxide concentration is 50 μL/mL to 200 μL/mL. Theoretically, the addition of hydrogen peroxide contributes to the rapid formation of a collagen gel film. However, experimental results show that, when a hydrogen peroxide concentration is higher than 200 μL/mL, hydrogen peroxide will be directly decomposed in the electrolyte to produce bubbles in the electrolyte, which is not conducive to the preparation of a uniform collagen gel film. For convenience of calculation, the concentration of hydrogen peroxide is expressed as a volume percentage of 5% to 17%. For example, various commercially-available hydrogen peroxide standards may be adopted, for example, the hydrogen peroxide standard solution used in the embodiment of the present application comes from Yonghua Chemical Co., Ltd. (Item No. 210401204).

Optionally, in S2, centrifugation is conducted at a centrifugal speed of 6,000 rpm/min to 8,000 rpm/min. This centrifugal speed contributes to excluding bubbles generated due to operations from a solution, thereby preventing a collagen gel film prepared subsequently from being non-uniform. The second collagen solution prepared in S2 needs to be slowly added to the electrolytic cell, which is intended to avoid bubbles generated due to a too-high viscosity of a solution.

Optionally, a voltage variation range in S4 is 0.22 V/cm² to 1.67 V/cm². The deposition time can be controlled at 10 min to 45 min, and the deposition time can be set and optimized according to parameters such as temperature, voltage, and an expected thickness of a collagen gel film. The EDP in S4 can be conducted at a constant voltage.

Optionally, the first collagen solution in S1 is prepared as follows: accurately weighing 400 mg of type I collagen, dissolving the type I collagen in 40 mL of UPW, and adding glacial acetic acid dropwise; thoroughly stirring a resulting system to promote the complete dissolution of the type I collagen, and adjusting a pH of a final solution to 1.5 to 3.0; and placing the solution in a dialysis bag (which does not allow the penetration of collagen, for example, Mw_{cut off}=7.0 kDa), placing the dialysis bag in a glacial acetic acid-containing aqueous solution, and conducting dialysis at 0° C. to 5° C. for 3 d to remove small-molecule impurities to obtain a collagen-containing viscous solution.

Optionally, in S2, 50 μL/mL to 100 μL/mL hydrogen peroxide is added to the first collagen solution obtained in S1, a resulting mixture is thoroughly stirred and then centrifuged at 5,000 rpm/min to 10,000 rpm/min and 0° C. to 5° C. to remove bubbles, and a collagen solution obtained after the centrifugation is stored in an ice-water mixed bath to prevent the decomposition of hydrogen peroxide.

Optionally, in S3, a titanium sheet is adopted as the cathode, and a platinum wire or a platinum sheet is adopted as the anode; and the second collagen solution obtained in S2 needs to be slowly added to the electrolytic cell carefully, which is intended to avoid bubbles generated due to a too-high viscosity of a solution.

Optionally, in S4, the electrodes are connected to an electrochemical workstation, a voltage is applied to the cathode, and the EDP is conducted for 500 s to 2,000 s at a constant current density of 0.5 mA/cm² to 10 mA/cm² and a voltage variation range of 0.22 V/cm² to 1.67 V/cm². The preparation time of the present application can be shortened to 8 min to 15 min, and within this time window, a collagen material with a thickness of about 300 μm in a wet state can be obtained, which facilitates the rapid preparation of a desired collagen material. The deposition time can also be adjusted according to a thickness of a desired collagen film.

Optionally, half reactions at the electrodes are as follows:

anode: 2H₂O−4e⁻→4H⁺+O₂; or, cathode: 4H₂O+4e⁻→4OH⁻+2H₂.

According to a second aspect of the present application, a collagen film is provided, where a preparation method of the collagen film includes:
dissolving a mixture including a collagen material in a solvent, and recycling to obtain the collagen film,
where the collagen material is selected from a collagen material prepared by the preparation method described above.

Optionally, the collagen film has a thickness of 180 μm to 550 μm.

Optionally, the collagen film has uniform appearance and is highly-transparent in both dry and wet states.

Optionally, the collagen film is obtained by linking short range-oriented collagen microfibers through non-covalent bonds.

Optionally, the collagen film has dense collagen arrangement.

Accordingly, the present application provides a collagen gel film, where the collagen gel film has very uniform and transparent appearance, dense collagen arrangement, and a uniform structure, is highly-transparent in both dry and wet states, and is obtained by linking short range-oriented collagen microfibers through non-covalent bonds; and the collagen gel film can be re-dissolved in a solvent and can be recycled. If the collagen gel film is not uniform inside (in particular, due to the generation of bubbles, defects occur inside the collagen gel film), the collagen gel film will undergo stress concentration and preferentially break at a structural defect under an external force. In addition, the structural defects occurring inside may cause the scattering of incident light, which reduces the transparency of the collagen gel film. After being prepared, the collagen gel film of the present application can be stripped from the electrode.

Optionally, the collagen gel film is prepared by the preparation method of a collagen material stripped from an electrode in the present application.

The present application provides a use of the collagen gel film or the preparation method thereof, and the use of the preparation method includes: preparing a collagen gel film on an electrode by the preparation method, and further acquiring a collagen material. The collagen material prepared by the present application can be re-dissolved in a solvent, and can be recycled by an EDP technology, that is, the recycling of the collagen material is allowed, which can promote the energy conservation and environmental protection.

The collagen gel film in the present application is produced on and stripped from the cathode, and according to a shape of the cathode, a heteromorphic collagen material of a same shape can be prepared. For example, in a preferred embodiment of the present application, when the cathode is a titanium tube, a hollow collagen tube may be obtained; and when a shape of the cathode is similar to a shape of a heart valve, a heart valve-like heteromorphic collagen material may be obtained.

The present application adopts an improved EDP technology to prepare a collagen material, where a collagen electrolyte is prepared with acetic acid as an acidity-regulating agent, a decomposition reaction of hydrogen peroxide is adopted as a cathode reaction, a Ti sheet is adopted as a cathode (namely, a working electrode), a Pt sheet is adopted as an anode (namely, a counter electrode), and the electrodes are arranged horizontally or vertically.

In the present application, UPW has an electrical resistivity of 18 MΩ*cm (25° C.), and in addition to water molecules, UPW includes almost no impurities, no mineral trace elements, no bacteria and viruses, and no organic matters such as chlorine-containing dioxins. During preparation of UPW, not only electroconductive media in water are almost completely removed, but also colloidal matters, gases, and organic matters that are not dissociated in the water are removed to a very low degree; and the preparation of UPW usually requires the four major steps of pretreatment, reverse osmosis (RO), ultra-purification, and post-treatment.

In the present application, constant-current deposition refers to an EDP process during which a current remains unchanged. For example, in a preferred embodiment of the present application, a current density during the EDP is 6.67 mA/cm².

In the present application, the heteromorphic collagen material means that collagen materials of various different shapes can be prepared according to needs, such as a rectangular collagen material, a round collagen material, a triangular collagen material, a trapezoidal collagen material, and an empty tube-shaped collagen material.

According to a third aspect of the present application, a use of a collagen material in preparation of a collagen film with highly-oriented and crystalline collagen fibers is provided. In the present application, the four steps of improved EDP assembly, mechanical stretching, ion incubation, and chemical crosslinking are adopted to prepare a collagen film that is mainly composed of long range-orientated collagen fibers with distinctive D-band characteristics and has a Young's modulus close to a Young's modulus of a native tendon. The collagen film material is obtained by further modifying a short range-oriented collagen material prepared on an electrode, and thus can be shaped according to a shape of the electrode. Electrodes of different shapes may also be adopted according to subsequent application requirements. For example, in a preferred embodiment of the present application, the prepared long range-oriented collagen film has similar properties to a native tendon, and has not only a similar crystal structure, but also a comparable Young's modulus.

A use of a collagen material in preparation of a collagen film with highly-oriented and crystalline collagen fibers is provided, including the following steps:
A1. stretching the collagen material in a length direction of the collagen material to produce a first collagen material;
A2. incubating a mixture including the first collagen material and phosphate-buffered solution (PBS) to obtain large-diameter collagen fibers; and
A3. subjecting the large-diameter collagen fibers to chemical crosslinking to obtain the collagen film with highly-oriented and crystalline collagen fibers,
where the collagen material is selected from a collagen material prepared by the preparation method described above.

Optionally, in the step A1, a strain degree of the stretching is Ts, and 50%≤Ts≤200%.

Optionally, in the step A2, a concentration of the PBS is 0.05 M to 0.5 M.

Optionally, in the step A2, the incubation is conducted for 6 h to 72 h.

Optionally, in the step A3, the chemical crosslinking includes photocrosslinking, glutaraldehyde crosslinking, genipin crosslinking, and polyphenol crosslinking.

Optionally, the photocrosslinking is conducted as follows: soaking the large-diameter collagen fibers in a 0.2 mg/mL to 3.0 mg/mL riboflavin solution, and allowing the photocrosslinking under ultraviolet (UV) irradiation for 1 d to 3 d.

Optionally, the glutaraldehyde crosslinking is conducted as follows: soaking the large-diameter collagen fibers in a 0.1% to 1% glutaraldehyde solution, and allowing the glutaraldehyde crosslinking for 10 min to 2 h.

Optionally, the genipin crosslinking is conducted as follows: soaking the large-diameter collagen fibers in a genipin solution with a mass percentage of 0.2% to 2.0%, and allowing the genipin crosslinking for 8 h to 14 h.

Optionally, the polyphenol crosslinking is conducted as follows: soaking the large-diameter collagen fibers in an aqueous solution of proanthocyanidin (PAC), tannic acid (TA), or gallic acid (GA) with a mass percentage of 0.1% to 2.0%, and allowing the polyphenol crosslinking for 8 h to 14 h.

Optionally, in the step A1, the stretched collagen material is soaked in ethanol to temporarily fix an oriented structure.

Optionally, in the step A2, two ends of the collagen material are fixed during ion incubation to make the collagen material undergo a continuous external action force without shrinking.

Optionally, the collagen film includes long range-orientated collagen fibers with distinctive D-band characteristics.

Optionally, the collagen film has a Young's modulus close to a Young's modulus of a native tendon.

According to a fourth aspect of the present application, a use of a collagen film in an artificial tendon is provided.

The use refers to a use of the collagen film obtained in the use described above in an artificial tendon.

According to an embodiment of the present application, a preparation method of the collagen film in the present application includes the following steps:
(1) mechanical stretching: taking a short range-oriented collagen microfiber film, and stretching the collagen microfiber film in a length direction of the collagen microfiber film, such that microfibers inside the collagen microfiber film are further oriented in a forced direction to produce a highly-oriented collagen material;
(2) ion incubation: subjecting the highly-oriented collagen material obtained in the step (1) to the ion incubation in 0.05 M to 0.5 M PBS for 20 h to 40 h, such that microfiber structures inside the highly-oriented collagen material are re-arranged to produce crystalline large-diameter collagen fibers with D-band characteristics; and
(3) chemical crosslinking: conducting photocrosslinking, glutaraldehyde crosslinking, or genipin crosslinking.

Optionally, in the step (1), the stretched E-Col collagen material is soaked in ethanol to temporarily fix an oriented structure. The strain degree of the stretching may be 50% to 200%, including 50% and 200%.

Optionally, in the step (2), two ends of the collagen material are fixed during ion incubation to make the collagen material undergo a continuous external action force without shrinking. For example, a tape is used to fix the two ends of the short range-oriented collagen film in a Petri dish or another container capable of holding a liquid. The incubation may be conducted for 24 h to 36 h. The incubation may be conducted at room temperature of 15° C. to 26° C.

In the present application, the PBS may be obtained by a conventional technique in the art. For example, the PBS may be obtained by fully dissolving 8 g of sodium chloride, 0.2 g of potassium chloride, 2.90 g of monosodium phosphate (MSP) dodecahydrate, and 0.2 g of monopotassium phosphate (MKP) in 1,000 mL of water. A too-high or too-low concentration of the salt solution will affect a speed at which a diameter of collagen fibers increases. Optionally, the concentration of the salt solution is 0.05 M to 1.0 M, where the unit refers to mol/L. A salt solution with a concentration of 0.1 M to 0.8 M and preferably 0.2 M to 0.5 M may also be adopted according to needs.

Optionally, the photocrosslinking is conducted as follows: soaking the highly-oriented and crystalline collagen film obtained in the step (2) in a 0.2 mg/mL to 3.0 mg/mL riboflavin solution, and allowing crosslinking under UV irradiation for 1 d to 3 d. The riboflavin solution includes 90% v/v ethanol-water as a solvent and has a concentration of 0.5 mg/mL to 2.0 mg/mL; and the crosslinking is conducted for 20 h to 40 h.

Optionally, the glutaraldehyde crosslinking is conducted as follows: soaking the highly-oriented and crystalline collagen film obtained in the step (2) in a 0.1% to 1% (volume percentage) glutaraldehyde solution, and allowing the glutaraldehyde crosslinking for 10 min to 2 h; and removing the residual glutaraldehyde component in the collagen film subsequently. The glutaraldehyde solution has a concentration of 0.5% w/v with 90% v/v ethanol-water as a solvent; and the crosslinking is conducted for 30 min to 50 min.

Optionally, the genipin crosslinking is conducted as follows: soaking the highly-oriented and crystalline collagen film obtained in the step (2) in a genipin solution with a mass percentage of 0.2% to 2.0%, and allowing crosslinking overnight; and removing the residual genipin component in the collagen film subsequently. Preferably, the genipin solution has a concentration of 0.5% to 1.0%; and the crosslinking is conducted for 8 h to 16 h.

Optionally, the polyphenol crosslinking is conducted as follows: soaking the highly-oriented and crystalline collagen film obtained in the step (2) in a PAC solution with a mass percentage of 1.0% to 2.0% and a pH of 8.5, and allowing crosslinking overnight; and removing the residual polyphenol component in the collagen film subsequently. Preferably, the PAC solution has a concentration of 1.5% to 2.0%; and the crosslinking is conducted for 12 h.

Optionally, the residual crosslinking agent component such as genipin or glutaraldehyde in the collagen film can be removed by repeatedly rinsing with UPW. For example, the residual crosslinking agent component may be removed by rinsing 3 to 5 times with UPW.

In the present application, UPW has an electrical resistivity of 18 MΩ*cm (25° C.), and in addition to water molecules, UPW includes almost no impurities, no mineral trace elements, no bacteria and viruses, and no organic matters such as chlorine-containing dioxins. During preparation of UPW, not only electroconductive media in water are almost completely removed, but also colloidal matters, gases, and organic matters that are not dissociated in the water are removed to a very low degree; and the preparation of UPW usually requires the four major steps of pretreatment, RO, ultra-purification, and post-treatment.

The EDP assembly (short range-orientation) leads to a collagen gel film with very uniform appearance, where the collagen gel film is highly-transparent in both dry and wet states, is obtained by linking short range-oriented collagen microfibers through non-covalent bonds, and has dense collagen arrangement, transparent appearance, and a uniform structure; and the collagen gel film can be re-dissolved in a solvent and can be recycled. The collagen gel film can be produced directly on an electrode, and can be stripped from the electrode after being prepared.

According to a fifth aspect of the present application, a use of a collagen solution in preparation of an artificial cornea is provided. The artificial cornea is a collagen-based artificial cornea that is highly-transparent and suturable and has a customizable structure (curvature and thickness). This technology includes the three steps of electrode design and customization, collagen assembly by an improved EDP technology, and chemical crosslinking. The collagen-based artificial cornea material is characterized in that the collagen-based artificial cornea material is composed of short range-oriented and densely-arranged collagen microfibers and has a transparency of 80% or more; and a macroscopic structure of the collagen-based artificial cornea material is a customizable arc structure, and a thickness of the collagen-based artificial cornea material can be adjusted according to electrochemical parameters of the improved EDP technology. The preparation process of the present application does not require the use of a complicated device, and the collagen-based artificial cornea material obtained can be used to replace or repair a natural cornea.

A use of a collagen solution in preparation of an artificial cornea is provided, including the following steps:
B1. acquiring a cathode with a curvature range of 7.8 to 8.5 as a working electrode of an electrolytic cell;
B2. placing a mixture including the collagen solution in the electrolytic cell, and conducting EDP to obtain a collagen material on the cathode; and
B3. subjecting the collagen material to chemical crosslinking to obtain the artificial cornea,
where the collagen solution is selected from the collagen solution in the preparation method described above.

Optionally, the EDP in the step B2 is conducted under the same conditions as the EDP described above.

Optionally, the chemical crosslinking in the step B3 is conducted under the same conditions as the chemical crosslinking in the step A3.

According to an embodiment of the present application, the method includes the steps of electrode design and customization, collagen assembly by an improved EDP technology, and chemical crosslinking, and can lead to a collagen-based artificial cornea with a shape consistent with a corneal shape.

Optionally, the electrode design and customization refers to the selection of a cathode with a curvature range of 7.8 to 8.5 as a working electrode.

Optionally, the collagen assembly by an improved EDP technology includes: adding hydrogen peroxide at a concentration of 5 μL/mL to 200 μL/mL to a collagen-containing acetic acid solution with a concentration of not higher than 20 mg/mL to obtain an electrolyte, arranging an anode and a cathode in parallel in the electrolyte with a distance of 0.5 cm to 3.0 cm between the anode and the cathode, and conducting EDP for 10 min to 60 min to obtain a collagen gel film deposited on the cathode.

Optionally, the cathode is matched with a natural corneal curvature.

Optionally, during the collagen assembly by an improved EDP technology, the hydrogen peroxide is added at a concentration of 5% to 17% (volume percentage).

Optionally, the chemical crosslinking is selected from photocrosslinking, glutaraldehyde crosslinking, or EDC-NHS crosslinking.

Optionally, the photocrosslinking is conducted as follows: soaking the collagen gel film in a 1 mg/mL riboflavin solution with 90% v/v ethanol-water as a solvent, and allowing crosslinking under 365 nm UV irradiation for 20 h to 30 h, for example, allowing crosslinking for 24 h to further strengthen the mechanical properties of the collagen gel film.

Optionally, the glutaraldehyde crosslinking is conducted as follows: soaking the collagen gel film in a 0.1% w/v to 0.6% w/v glutaraldehyde solution with 90% v/v ethanol-water as a solvent, allowing crosslinking for 15 min to 50 min, and removing the residual glutaraldehyde component in the collagen film subsequently. Preferably, the glutaraldehyde solution has a concentration of 0.2% w/v to 0.5% w/v. The residual glutaraldehyde component can be removed by rinsing. For example, the collagen gel film can be repeatedly rinsed with UPW to remove the residual glutaraldehyde component in the collagen film.

Optionally, the EDC-NHS crosslinking is conducted as follows: soaking the collagen gel film in a 90% v/v ethanol-water solution of EDC/NHS (n(EDC):n(NHS)=2:1) in which a mass concentration of EDC is 0.5 g/L, adjusting a pH with an MES buffer to 5.5, and allowing crosslinking at 4° C. for 24 h; and taking the collagen gel film out, and repeatedly rinsing the collagen gel film with deionized water.

Accordingly, the present application provides a novel artificial cornea, where the artificial cornea is matched with a natural corneal curvature, is highly-transparent and suturable, and has a customizable structure. Optionally, the artificial cornea is prepared by the method described above.

According to a sixth aspect of the present application, a use of an artificial cornea in cornea repair is provided.

The use of an artificial cornea in cornea repair includes the following steps:
D1. cutting the artificial cornea;
D2. filling and suturing a cut artificial cornea to a corneal defect site; and
D3. allowing repair for at least 2 weeks,
where the artificial cornea is selected from the artificial cornea obtained in the use described above.

The present application provides a use of a preparation method of the artificial cornea, including: preparing an artificial cornea by the preparation method of the artificial cornea, and using the artificial cornea to replace or repair a natural cornea.

Optionally, the artificial cornea has one or more of the following characteristics: the artificial cornea is matched with a corneal curvature, a thickness of the artificial cornea can be designed according to needs, and the artificial cornea has an excellent optical transparency, an excellent microscopic morphology, a high mechanical strength, suture resistance, and excellent cell compatibility, which is conducive to improving the adhesion, proliferation, and migration of corneal epithelial cells (CECs).

Optionally, the use includes the following steps:
(A) cutting the artificial cornea as needs;
(B) filling and suturing a cut artificial cornea to a corneal defect site; and
(C) allowing repair for at least 2 weeks.

Optionally, a diameter of the artificial cornea can be designed according to an area of the corneal defect site, such as 6 mm or more and 7 mm or more. The repairing time should usually be determined according to conditions of the corneal defect site, and can be no less than 3 weeks to 6 weeks or even 8 or more weeks, for example.

In contrast to the EDC crosslinking technology directly for collagen molecules that is reported previously (A Simple, Cross-linked Collagen Tissue Substitute for Corneal Implantation, Investigative Ophthalmology & Visual Science, May 2006, Vol. 47, No. 5, 1869-1875), in the present application, collagen molecules are assembled in advance by the improved EDP technology to produce collagen microfibers, and then the collagen microfibers are chemically crosslinked, which leads to excellent mechanical properties and a controlled curvature and thickness, and involves a simple and efficient preparation process.

Compared with the previous cyclodextrin technology (CN106456833A), the preparation process of the present application is rapid and simple, allows a customizable macroscopic structure and a controllable thickness, and does not require the incorporation of cyclodextrin in a material, which avoids the subsequent problem of ocular tissue compatibility.

According to a seventh aspect of the present application, a use of a collagen material in preparation of a bandage is provided. The bandage has a high strength and toughness, and can automatically return to a soft state and dynamically relax in vivo.

A use of a collagen material in preparation of a bandage is provided, including the following step:
placing a mixture including the collagen material in a salt solution to obtain the bandage,
where the collagen material is selected from a collagen material prepared by the preparation method described above.

Optionally, the salt solution is a solution of a soluble salt of a Hofmeister ion. The salt solution is a first salt solution.

Optionally, the Hofmeister ion is selected from at least one of CO₃²⁻, SO₄²⁻, S₂O₃²⁻, H₂PO₄⁻, NO₃⁻, CH₃COO⁻, ClO₄⁻, F⁻, Cl⁻, and Br⁻.

Optionally, a concentration of the salt solution is 0.1 M to 4 M.

Optionally, the mixture is placed in the salt solution for 0.5 h to 60 h.

Optionally, the bandage includes a collagen film with a high strength and a high toughness of the Hofmeister ion; and the collagen film can gradually return from a high-strength state to a soft state in an environment. The high strength refers to a breaking strength of 2.0 MPa or more or a Young's modulus of 9.0 MPa or more; and the high toughness refers to a toughness value of 0.5 MJ/M³ or more.

Optionally, an environment in which a mechanical state of the collagen film changes is a solution, such as pure water or a salt and/or enzyme-containing body fluid; or the environment may also be a simulated human body fluid in an embodiment. In a preferred embodiment, in collagenase (100 U/mL)-containing water at 37° C., the collagen film can be completely degraded within about 45 h. Optionally, the bandage has a thickness of 50 μm to 1,000 μm and preferably 300 μm to 800 μm, such as 100 μm, 200 μm, 300 μm, 400 μm, 500 μm, 600 μm, 700 μm, 800 μm, and 900 μm.

The preparation method of the collagen bandage in the present application includes the two stages of assembly by an EDP technology and soaking by the Hofmeister ion:
preparing a collagen solution, and conducting EDP with the collagen solution to obtain a short range-oriented collagen gel film that can be stripped from an electrode; and
soaking the collagen gel film in a salt solution for 8 h to 60 h.

A salt can precipitate a protein from an aqueous solution, and this effect is known as a Hofmeister effect. A principle of the Hofmeister effect is as follows: a direct interaction of salt ions with macromolecules and hydrated shells thereof leads to the removal of hydrated water of a protein, thereby causing the folding and precipitation of the protein. Hofmeister series ions can be used to strengthen a weak interaction within an E-Col network to strengthen the mechanical properties of the film.

The present application adopts the two stages of collagen assembly by the improved EDP technology and soaking by a Hofmeister ion to prepare a high-strength and high-toughness collagen film material. The collagen film E-Col prepared by the improved EDP technology is mainly based on assembly through some non-covalent bonding, such as hydrogen bonding and hydrophobic interactions, which ensures that an internal structure of the collagen film E-Col has a dynamic remodeling ability (because the collagen film is easily broken and regenerated due to non-covalent bonding).

If not treated in any way, the electrochemically-assembled collagen film E-Col has a breaking strength of 0.13 MPa, a Young's modulus of 0.32 MPa, and a toughness value of 0.19 MJ/M³. A conventional idea is to improve the mechanical properties of a material through chemical or physical crosslinking. When chemical crosslinking is conducted with 0.5% w/v glutaraldehyde, the breaking strength can be increased to 5.22 MPa, the Young's modulus can be increased to 11.39 MPa, and the toughness value can be increased to 1.27 MJ/M³; and when UV photocrosslinking is conducted with 1 mg/mL riboflavin, the breaking strength can be increased to 1.33 MPa, the Young's modulus can be increased to 1.24 MPa, and the toughness value can be increased to 0.23 MJ/M³. However, compared with the Hofmeister ion strengthening technology of the present application, the crosslinking technology can only improve the mechanical properties to a limited extent. More importantly, due to the introduction of a large number of covalent bonds inside the collagen film, the collagen E-Col obtained after crosslinking will not have a dynamic recovery ability. A collagen film prepared by the preparation method of the present application can have a breaking strength of 5.85 MPa or more, a Young's modulus of 16.42 MPa or more, and a toughness value of 3.33 MJ/M³ or more.

Impacts of different ions on the solubility of a protein are significantly different, where an impact of an anion is more significant than an impact of a cation, and hydration effects (that is, effects to remove hydrated water of a macromolecule) of anions themselves are different. Generally, anions capable of changing the solubility of a protein are CO₃²⁻, SO₄²⁻, S₂O₃²⁻, H₂PO₄⁻, NO₃⁻, CH₃COO⁻ClO₄⁻, F⁻, Cl⁻, Br⁻, SCN⁻, I⁻, and the like. However, not all Hofmeister anions are suitable for the collagen film of the present application. For example, when the collagen film is soaked in a NaCl solution, the collagen film remains a soft state without obvious mechanical strengthening; when the collagen film is soaked in a NaI solution, the collagen film will swell; and when the collagen film is soaked in a NaSCN solution, the collagen film will be directly dissolved. As a comprehensive consideration, the selection of CO₃²⁻ or SO₄²⁻ will lead to a significant effect.

Optionally, a second salt solution is used instead of the first salt solution, and the second salt solution is selected from a solution of a CO₂²⁻ or SO₄²⁻-containing soluble salt. For example, a salt is selected from one or more of ammonium sulfate, sodium sulfate, or sodium carbonate.

According to an eighth aspect of the present application, a bandage is provided.

The bandage includes a short range-oriented collagen film, where the bandage is selected from the bandage obtained in the use described above.

Optionally, the collagen film is capable of automatically returning to a soft state and dynamically relaxing.

Optionally, the collagen film has a breaking strength of 2.0 MPa to 8 MPa.

Optionally, the collagen film has a Young's modulus of 9.0 MPa to 18.0 MPa.

Optionally, the collagen film has a toughness value of 0.5 MJ/M³ to 5.5 MJ/M³.

Optionally, the bandage has a thickness of 50 μm to 1,000 μm.

According to a ninth aspect of the present application, a use of the bandage in artery banding is provided.

A use of the bandage described above in artery banding is provided.

Optionally, the use includes the following steps:
E1. determining a position for artery banding;
E2. wrapping the bandage around the position for the artery banding, and making a sliding hydrogel knot; and
E3. adjusting a degree of the artery banding, and removing an excess part of the bandage.

The collagen bandage prepared in the present application can be dissolved automatically and gradually relax in vivo or in a simulated body fluid (SBF). In some intervention surgeries (such as pulmonary artery banding (PAB)), a medical bandage is wrapped around an artery to temporarily restrict a blood flow, thereby protecting a vulnerable site downstream from being damaged by a high blood pressure. After a surgery, the bandage should provide a sustained ability to constrict a blood vessel in a short term, but after a heart function gradually recovers over time, the bandage should gradually relax to allow a normal blood flow (a recovery time depends on clinical details). Therefore, a desired material for such a medical bandage should have an ability to dynamically relax in vivo. The E-Col film of the present application can meet the requirements of these mechanical properties. When the E-Col film of the present application is in a body, because the salt can gradually ooze from a network of collagen fibers, a mechanical strength of the E-Col will gradually decrease, such that a banding effect for an implantation site is weakened.

The SBF is a liquid simulating a composition and pH of a human body fluid. In a preferred embodiment of the present application, a product with Item No. PH1820 from Guangzhou Yazhi Biotechnology Co., Ltd. is adopted.

Optionally, a method for using the bandage as an artery banding bandage is as follows:
determining a position for artery banding; wrapping the collagen bandage of the present application around the position for the artery banding, and making a sliding hydrogel knot; and adjusting a degree of the artery banding, and removing an excess part of the bandage. A method to make the sliding hydrogel knot can be as follows: with one end of a hydrogel band as an axis, tying the other end around the axis to form a surgical knot, making the axis pass through a center of the surgical knot to form a basic knot, and tightening the knot.

The present application uses the first salt solution for soaking, where bound water inside collagen is deprived through a strong hydration effect to create a hydrophobic microenvironment inside the collagen, which can strengthen the H bonding and hydrophobic interactions among collagen microfibers (water will interfere with the H bonding and hydrophobic interactions), thereby greatly improving the mechanical strength and toughness of a collagen film. Experimental results have shown that the high-toughness collagen material of the present application can maintain a fixed shape, and will not be ruptured when lifting a heavy object of 1 kg or stretched during knotting. In an in vivo environment, with the dissolution of a Hofmeister salt, the mechanical properties of the collagen film will decrease, such that the collagen film loses its restriction effect for a blood flow and is eventually degraded by collagenase in vivo. Therefore, the collagen film can be used as an artery banding bandage in vivo to provide a temporary restriction effect for a blood flow rate.

Possible beneficial effects of the present application:
(1) Because acetic acid is a weak acid, a specified buffering capacity can be provided to make a pH near a cathode close to an isoelectric point of collagen, such that a collagen material can be obtained directly on a surface of the cathode.
(2) A shape of the cathode can be changed to facilitate the preparation of collagen materials of various heteromorphic structures.
(3) A cathode reaction is a decomposition reaction of hydrogen peroxide, and does not produce bubbles, which is conducive to acquisition of a collagen material with very uniform and dense appearance, and can also solve the problem that a prepared collagen film may have a thin upper part and a thick lower part due to a gravity especially when horizontal electrodes are adopted.
(4) A distance between two electrodes can be controlled at a centimeter grade, which is convenient to operations.
(5) A preparation time is as short as 10 min to 15 min.
(6) The collagen material has the following characteristics: The collagen material is composed of short range-oriented amorphous collagen microfibers, and has dense collagen arrangement, transparent appearance, and a uniform structure, which is conducive to acquisition of excellent mechanical and optical properties; the collagen material has a plastic deformation ability; and collagen microfibers in the collagen material are linked through non-covalent bonds, and thus the collagen material can be re-dissolved in a solvent and can be recycled by an EDP technology, that is, recycling of the collagen material is allowed.
(7) Mechanical stretching is adopted to produce long range-oriented collagen microfibers.
(8) Ion incubation is conducted in a stretched state to obtain a long range-oriented and crystalline collagen material.
(9) The improved EDP technology is adopted to shape a collagen material according to a shape of an electrode, such that it is not just limited to a collagen beam.
(10) The collagen material of the present application has D-band characteristics, and is highly similar to a native tendon in terms of characteristics such as appearance, microscopic morphology, Young's modulus, and crystal structure.
(11) The preparation method of the present application involves simple operations, does not require complicated instruments, and can quickly and effectively prepare a collagen film material that is highly similar to a native tendon or ligament.

### BRIEF DESCRIPTION OF THE DRAWINGS

FIGS. 1A-1B are schematic diagrams of an assembly process of the EDP technology, where electrodes are arranged in the following two ways: 1. the two electrodes are vertically arranged in parallel in an electrolytic cell, as shown in FIG. 1A; and 2. the two electrodes are horizontally arranged in parallel in an electrolytic cell, as shown in FIG. 1B;

FIGS. 2A-2B show collagen gel films, where FIG. 2A shows a collagen gel film appearing on a cathode; and FIG. 2B shows that the E-Col collagen material has very uniform appearance and is highly-transparent in both dry and wet states;

FIGS. 3A-3B show test results of optical properties of collagen, where the E-Col exhibits a high optical transparency, which is of nearly 90% in a wavelength range of 450 nm to 780 nm (visible light range), as shown in FIG. 3A; and the collagen gel film also has a very low haze, which is merely of 10% in the visible light range, as shown in FIG. 3B;

FIGS. 4A-4B show micromorphological characterization results of collagen, where it can be seen that the E-Col film has a dense organizational structure and a density of 0.88 g/cm³, and there is the oriented arrangement of nanofibers at both a surface and a cross section; in contrast, the S-Col film assembled in a solution has a loose network inside and has a density of 0.45 g/cm³, and coarse fibers with a diameter of about a few micrometers are randomly aggregated in the film, as shown in FIG. 4A; transmission electron microscopy (TEM) images in FIG. 4B show that the E-Col film is obtained through tight arrangement of fine microfibers, and high-magnification TEM images reveal that the microfibers have a diameter of about 10 nm and do not have significant D-band characteristics of collagen fibers; and in contrast, there are loosely-arranged fibers at a micro-scale in the S-Col film, and high-magnification TEM images show that micro-scale fibers in the S-Col film are fibrils with a diameter of 50 nm and the fibrils have significant D-band characteristics of type I collagen, where a D band is at about 64.5 nm;

FIGS. 5A-5F show orientation characterization results, where polarized optical microscopy (POM) images in FIGS. 5A-5B show that the S-Col film has no obvious optical birefringence phenomenon, which is manifested as an isotropic structure; and an optical birefringence phenomenon is observed in some regions of the E-Col gel film, indicating an oriented structure in these regions; small-angle X-ray scattering (SAXS) data in FIGS. 5C-5D show that a two-dimensional (2D) SAXS pattern of the S-Col has a ring with an almost consistent intensity, indicating an isotropic structure; and a 2D SAXS pattern of the E-Col has a significantly-elongated ring, indicating an anisotropic structure; in ID-SAXS profiles in FIG. 5E, when a q value is in a range of 0.2 nm⁻¹ to 1.2 nm⁻¹, the S-Col has a significant D-band characteristic scattering peak in a 1D-SAXS profile (a D band calculated by the Bragg equation is at about 62.7 nm), and in contrast, the E-Col does not have a significant D-band characteristic peak, indicating an amorphous structure; and in 1D-SAXS profiles in FIG. 5F, when a q value is in a range of 0.05 nm⁻¹ to 0.4 nm⁻¹, the E-Col film shifts to a higher q value than the S-Col film, indicating the tightened arrangement of fibers;

FIGS. 6A-6C show characterization results of dynamic and static mechanical properties of a collagen material prepared by the EDP technology, where after being stretched, the collagen film obtained in Example 1 undergoes a significant plastic deformation; and after a stretching force is removed, the collagen film does not return to its original shape, as shown in FIG. 6A; static mechanical test results show that a stretching rate is set at 10 mm/min to obtain a stress-strain curve of the collagen film; and the E-Col gel film has a Young's modulus of 0.32±0.11 MPa (allowing a large deformation), an elongation at break of 220.41±5.07%, and a tensile strength of 0.13±0.03 MPa; the E-Col gel film undergoes a stress yield in a very small region, indicating that there is merely a weak crosslinking mechanism inside, as shown in FIG. 6B; and dynamic mechanical test results show that the E-Col film undergoes a large deformation, and there is a significant lag between loading and unloading cycles, indicating viscoelasticity, as shown in FIG. 6C;

FIGS. 7A-7B show test results of reversibility of a collagen film, where when the collagen film E-Col prepared in Example 1 is soaked in a 0.1 M acetic acid solution with a pH of 3.5 or a 0.1 M urea solution, the collagen film E-Col is rapidly dissolved within less than 10 min; and in contrast, the S-Col remains stable persistently, indicating that the intermolecular binding in the E-Col film mainly depends on some weak molecular interactions, and a solution of the E-Col film dissolved by acetic acid can undergo EDP once again to obtain an E-Col material;

FIG. 8 shows controllable preparation of a collagen film, where a thickness of the collagen film is controlled by changing a constant current density and a deposition time; and when the deposition time varies from 0 s to 3,000 s, the thickness of the collagen film can vary from 0 μm to 400 μm at a current density of 2.5 mA/cm², the thickness of the collagen film can vary from 0 μm to 450 μm at a current density of 5 mA/cm², and the thickness of the collagen film can vary from 0 μm to 550 μm at a current density of 10 mA/cm²;

FIGS. 9A-9B show pictures of collagen materials with different macroscopic geometric shapes, where a shape of a cathode can be changed, or a titanium tube or a stainless steel heteromorphic column with a valve-shaped end is used as a cathode to prepare various heteromorphic structural materials; when the titanium tube is adopted as the cathode, a hollow collagen tube may be obtained, as shown in FIG. 9A; and when a shape of the cathode is similar to a shape of a heart valve, a heart valve-like heteromorphic collagen material may be obtained, as shown in FIG. 9B;

FIG. 10 is a schematic diagram of the preparation method of a collagen film with a highly-oriented and crystalline collagen fiber structure in the present application, where the preparation method mainly includes mechanical stretching, ion incubation, and chemical crosslinking of E-Col;

FIGS. 11A-11D show the appearance of collagen materials under different tensile forces and a control, where FIG. 11A shows the appearance of a collagen film in each group; FIG. 11B shows observation results of POM, and it can be seen that an S-Col film in a control group has no obvious optical birefringence phenomenon, which is manifested as an isotropic structure; there is an optical birefringence phenomenon in some regions of an E-Col film that is not stretched and deformed, indicating an ordered structure in these regions; and when an E-Col film is stretched to a high strain degree, an obvious optical birefringence phenomenon can be observed throughout the entire region of the E-Col film, and when a deformation degree is further increased to 200%, there is a bright birefringence color, indicating the formation of a highly-oriented structure in the E-Col film; TEM images in FIG. 11C show that the S-Col film in the control group has a loose isotropic structure (red circles indicate fibrils perpendicular to a cross section); a density and an orientation degree of the E-Col film can be significantly improved through mechanical stretching; and the greater the deformation induced by mechanical stretching, the denser the arrangement of internal microfibers and the higher the orientation degree; and 2D SAXS patterns in FIG. 11D show that a 2D SAXS pattern of the S-Col film in the control group has a ring with an almost consistent intensity, which is consistent with an internal isotropic structure; and a 2D SAXS pattern of the E-Col film has a significantly-elongated ring, indicating the presence of an anisotropic nanofiber structure;

FIGS. 12A-12B show test results of an orientation degree of stretching of an E-Col film, where FIG. 12A shows azimuthal-integrated intensity distribution curves, and the results show that an azimuthal-integrated intensity distribution curve of the E-Col film gradually narrows with the increase of a strain degree; and a Herman's orientation parameter (f_{c}) is a quantitative index describing an orientation degree and can be calculated according to an azimuthal-integrated intensity distribution curve; and FIG. 12B shows that an orientation degree of an S-Col film in a control group is almost 0 (f_{c}=0.02), and an E-Col film that is not stretched has a low orientation degree (f_{c}=0.15); and with the increase of a tensile strain of the E-Col film, the Herman's orientation parameter gradually increases from f_{c}=0.15 to f_{c}=0.93 (when a strain is 200%), indicating that the stretching of the E-Col film will induce the formation of an oriented structure in a strain direction;

FIGS. 13A-13D show the comparison of macroscopic and microscopic structures between an E-Col film and a native tendon, where FIG. 13A and FIG. 13C show macroscopic and microscopic images of the E-Col film, respectively, and FIG. 13B and FIG. 13D show macroscopic and microscopic images of the native tendon, respectively; as shown in FIG. 13A, after mechanical stretching and ion incubation, the E-Col film that is highly-transparent initially becomes milky-white and opaque and has millimeter-scale-oriented stripes on its surface, that is, the E-Col film becomes similar to the native tendon (FIG. 13B), which may be attributed to a change of an optical transparency caused by the formation of higher-order hierarchical structures (namely, large-diameter fibers); and FIG. 13C and FIG. 13D show low-magnification and high-magnification SEM images, and it can be seen that, after PBS incubation, the E-Col film presents higher-order hierarchical structures, that is, densely-arranged fibers with a diameter of 5 μm to 10 μm;

FIGS. 14A-14C show characterization results of an orientation degree and a crystal form of an E-Col film, where it can be seen from a 2D SAXS pattern in FIG. 14A that, after 200% pre-stretching and ion incubation, the E-Col film still has a significantly-elongated ring, indicating that an anisotropic structure still retains after the ion incubation, and f_{c} is calculated to be about 0.52 to 0.53; and there is a significant D-band diffraction ring in the 2D SAXS pattern, indicating that collagen molecules are orderly arranged after the ion incubation; a 2D SAXS pattern of a native tendon shown in FIG. 14B also has a significant D-band diffraction ring, and f_{c} is calculated to be about 0.69 to 0.72; and it can be seen from IDSAXS profiles in FIG. 14C that, after the ion incubation, a similar crystal structure to the native tendon is produced in the E-Col film;

FIGS. 15A-15B show characterization results of static mechanical properties of an E-Col film, where FIGS. 15A-15B show a breaking stress and a Young's modulus of an E-Col material in a dry state, respectively; the E-Col material has a breaking stress of about 108+6 MPa, which is slightly lower than a breaking stress of a native tendon (128+14 MPa); and the E-Col material has a Young's modulus of 0.795±0.060 GPa, which basically reaches a level (0.890±0.118 GPa) of a native tendon;

FIGS. 16A-16B are schematic diagrams of a collagen material assembled by an EDP technology and a preparation method thereof, where FIG. 16A shows a collagen gel film prepared on an electrode with a fixed curvature, and the collagen gel film can be used as an artificial cornea; and FIG. 16B shows that a collagen gel film presents different thicknesses with the adjustment of preparation parameters;

FIGS. 17A-17C show test results of properties of a collagen material prepared by an EDP technology after chemical crosslinking, where as shown in FIGS. 17A-17B, compared with a collagen film S-Col assembled in a solution, the E-Col collagen gel film assembled by an improved electrochemical technology exhibits a high transmittance of 80% to 90% in a wavelength range of 380 nm to 800 nm, and the transmittance increases with the increase of a wavelength (a transmittance of a healthy human cornea at 430 nm is about 80%, and a transmittance of the normal human cornea at 500 nm or more can be close to 100%); and when an E-Col gel material is crosslinked by different methods, an optical transparency of the E-Col gel material basically does not change, which is significantly better than an S-Col gel material assembled in a solution; and in FIG. 17C, a current intensity and an application time of the current intensity are controlled to prepare E-Col collagen films with a fixed curvature that have thicknesses of about 200 μm, 300 μm, 400 μm, and 500 μm in a gel state, respectively, and test results show that the increase of a thickness basically does not affect the high transmittance and low haze of the material;

FIG. 18 shows characterization results of a microscopic morphology of a collagen material prepared by an EDP technology after chemical crosslinking, where the S-Col, E-Col-UV, E-Col-GA, and E-Col collagen films prepared in Example 28 each are lyophilized, and then microscopic morphologies of the lyophilized collagen films are analyzed by SEM (S-4800, Hitachi); the S-Col film is milky-white and translucent, and has coarse fibrous structures on its surface and a morphology of loosely-stacked fibers at a cross section; the E-Col gel materials are highly-transparent; a surface morphology of an E-Col gel material shows the oriented arrangement of small-size fibers, and a cross-section morphology of an E-Col gel material shows the tight stacking of layered structures; after crosslinking, a surface of the material can still retain an oriented structure well, and it can be seen from a cross-sectional structure that the crosslinking increases the structural compactness to some extent; and structural observation results further prove that, because E-Col can retain an excellent microscopic morphology after crosslinking, E-Col also exhibits excellent optical properties at a macroscopic level;

FIGS. 19A-19D show characterization results of mechanical properties of a collagen material prepared by an EDP technology after chemical crosslinking, where E-Col-UV, E-Col-GA, and E-Col collagen films are prepared by the same method as in Example 28 and cut into rectangular sample strips each with a length of 30 mm and a width of 10 mm, and an Electro-Force 3200 biodynamic tester is used to compare mechanical properties of the collagen films at room temperature; as shown in FIGS. 19A-19B, a stretching rate is set at 10 mm/min to obtain stress-strain curves of the collagen films; compared with the E-Col gel film, the E-Col-UV and E-Col-GA crosslinked gel films have a significantly-increased strength, but a decreased elongation at break; although a breaking strain decreases after crosslinking, breaking strengths of E-Col-UV (1.33±0.19 MPa) and E-Col-GA (5.22±0.73 MPa) are significantly improved; and it is speculated that E-Col-GA has a high mechanical strength after crosslinking due to a high crosslinking density, indicating that E-Col-GA can withstand high external shearing and stretching during a surgery and a postoperative repair to maintain the mechanical stability of the material and an affected site; FIG. 19C shows test results of suture strengths of the materials, and the test results show that E-Col-GA has higher suture resistance than E-Col-UV; and FIG. 19D shows quantification results of suture resistance of the materials, and the quantification results show that the suture resistance (1.75±0.3 N) of E-Col-GA is significantly higher than the suture resistance (0.33±0.13 N) of E-Col-UV, indicating that the suture resistance of E-Col-GA is strong enough to allow implantation with a suture line;

FIGS. 20A-20C show results of cell adhesion and proliferation tests of human corneal epithelial cells (HCECs) on an E-Col-GA film, where the E-Col-GA gel film is selected for the cell experiments; it can be seen from FIG. 20A that HCECs can well adhere to a surface of the E-Col-GA film in a spreading state; and HCECs inoculated on the E-Col-GA film and a tissue culture well plate undergo sustained proliferation on day 1, day 3, and day 5, and no significant dead cells are found during the 5-day observation period; FIG. 20B shows that the E-Col-GA film has excellent cell compatibility, and this observation result is confirmed by a cellular metabolism activity quantified by CCK-8, where HCECs inoculated on the E-Col-GA film and the tissue culture well plate control exhibit high viabilities (higher than 90%) on day 1, day 3, and day 5 after inoculation; as shown in FIG. 20C, the E-Col-GA film has excellent cell compatibility and can support the adhesion and proliferation of HCECs;

FIGS. 21A-21B show cell scratch assay results and a statistical chart thereof, where FIG. 21A shows that the migration of epithelial cells inoculated on a surface of the E-Col-GA gel film can be completed within less than 36 h to fill a scratch region (with a width of about 500 μm), and in contrast, epithelial cells migrate slowly at a bottom of the well plate and still fail to fill a scratch region at 36 h; and to quantify the migration of cells to a scratch region, a percentage of an area occupied by cells migrating to the scratch region in an initial area of the scratch region is calculated at different time points, and results show that, at 12 h, 24 h, and 36 h after scratching, cell migration completion rates on the E-Col-GA gel film are significantly higher than corresponding cell migration completion rates on the control (tissue culture well plate); and as shown in FIG. 21B, a cell migration completion rate on the E-Col-GA gel film at 36 h basically reaches 100%, which is 33% higher than a corresponding cell migration completion rate on the control (tissue culture well plate), indicating that the E-Col-GA film is conducive to the migration of HCECs;

FIGS. 22A-22C show results of a transplantation and repair test of an E-Col-GA film at a corneal lamella in vivo, where a schematic diagram of a healthy cornea, a cornea with a defect (diameter: 7 mm, and depth: 250 μm) constructed, and a cornea with E-Col-GA transplanted and actual pictures thereof during a surgery are illustrated; and it can be seen from the actual pictures that E-Col-GA can be sutured to a defect site while presenting a high-transparency character;

FIGS. 23A-23C show examination results of postoperative slit lamp bioscopy, where 1, 2, 4, 6, and 8 weeks after a surgery, corneal tissues of rabbits under general anesthesia are subjected to non-destructive observation under a slit lamp; as shown in FIG. 23A, 1 week after the surgery, in the implanted material-free blank group, an obvious edema occurs to cause a specified degree of turbidity of a cornea, and the edema gradually disappears over time to recover a transparent state, but a distinct defect boundary (marked by a white arrow) can still be observed after the 8-week observation period is completed; in contrast, in an experimental group (E-Col-GA) and a positive control group (commercial acellular porcine cornea matrix), after a material is implanted, a specified immune response first occurs, including emergence of some fine blood vessels around the material and a slight edema of a tissue, which is a normal immune response after the material is implanted to a corneal defect site, but the blood vessels generated under stimulation gradually disappear over time; FIG. 23A shows that there is a significant migration of epithelial cells in different groups (an epithelial defect is stained green by fluorescein), but epithelization rates are different; and in a blank control group, a migration rate of CECs is the highest because CECs grow along a surface of a defective stromal layer; FIG. 23B shows that the experimental group (E-Col-GA) exhibits a comparable or even higher corneal epithelization rate to or than the positive control group (acellular porcine cornea matrix), and undergoes almost complete epithelization 4 weeks after the surgery; and in FIG. 23C, a proportion of an area of a vascularized region after implantation is quantitatively calculated by the software Image J, and it can be seen that obvious blood vessels are generated within 2 weeks after implantation in both the experimental group and the positive control group, but the experimental group has a smaller vascularized region than the positive control group, and two weeks after implantation, blood vessels initially generated in the two groups gradually disappear; 6 weeks after implantation, blood vessels initially generated in the experimental group completely disappear, while there are still some blood vessels that do not disappear in the positive control group; 8 weeks after implantation, a cornea of the experimental group basically becomes completely transparent, but the positive control group still has a specified degree of turbidity and is not restored to a normal transparency of a cornea, which is presumably related to a significant immune response after implantation; and compared with the blank control group, no obvious defect boundary can be observed in both implanted groups after the 8-week repair period is completed;

FIGS. 24A-24C show examination results of postoperative optical coherence tomography (OCT), where FIG. 24A shows tomographic images of defects in different groups 0 to 8 weeks after a surgery, and it can be seen that, in the implanted material-free group, an obvious edema occurs 1 week after the surgery, and 2 weeks after the surgery, the edema basically disappears, but an obvious defect still can be observed on a corneal stromal layer; although complete epithelization can be observed 4 weeks after the surgery, a thickness of the corneal stromal layer at a defect basically cannot return to a normal level even 8 weeks after the surgery (as shown by a white arrow in an image); in contrast, an experimental group and a positive control group both are basically completely restored to a thickness of a healthy cornea one day after the surgery; one day after the surgery, an interface between a material and a stromal layer can be observed (as shown by an orange arrow in an image), and 1 week after the surgery, the interface between the material and the stromal layer has gradually blurred (as shown by a red arrow in an image), indicating the fusion of the material with the autologous stromal tissue; the gradual epithelization can be observed during the fusion of the material with the autologous stromal tissue, and the generation of a complete epithelialized tissue can be observed 8 weeks after implantation (as shown by a white arrow in an image); and a region indicated by a red arrow also shows the basic fusion of the material with the autologous stromal layer; and it can be seen from FIGS. 24B-24C that a healthy cornea of a rabbit has a thickness of about 550 μm; after a defect is constructed, the thickness of the cornea decreases significantly, and a measured thickness of a defective cornea is about 200 μm; 8 weeks after the surgery, a thickness of a cornea in the implanted material-free group increases to some extent; and in contrast, in the experimental group and the positive control group, the thickness of the healthy cornea is basically restored 8 weeks after implantation;

FIGS. 25A-25E show test results of mechanical strengthening for an E-Col film based on a Hofmeister effect, where in FIG. 25A, E-Col and S-Col films each are cut into rectangular sample strips each with a length of 30 mm and a width of 10 mm, and then soaked in a (NH₄)₂SO₄ (2 mol/L) solution for 24 h; FIG. 25B shows that a network of the E-Col collagen film can undergo a significant stiffening phenomenon under classical stimulation of Hofmeister salt-ammonium sulfate, which significantly strengthens the mechanical properties of the collagen film; FIGS. 25C-25D show that, after an E-Col network is treated with (NH₄)₂SO₄ (2 M, 24 h), a resulting transparent E-Col gel film can withstand a load of 1 kg while retaining the flexibility of the network, and the film can be knotted without rupturing; and in contrast, after an S-Col network is treated by the same method, a small strengthening effect is allowed, and a treated film cannot withstand a load of 500 g and is prone to brittle fracture; and FIG. 25E shows states of the E-Col gel film soaked in different Hofmeister salts for 24 h;

FIGS. 26A-26C show quantitative characterization results of mechanical properties of an E-Col film strengthened based on a Hofmeister effect, where a collagen gel film is soaked in ammonium sulfate solutions with different concentrations (1 M, 2 M, 2.5 M, and 4 M) at room temperature for 12 h to strengthen the hydrophobic and H bonding interactions, and then an Electro-Force 3200 biodynamic tester is used to investigate the mechanical properties of the collagen film at room temperature; and a sample is stretched by a fixture at a strain rate of 10 mm/min, a Young's modulus (MPa) of the sample is calculated based on a slope factor of an initial linear region of a stress-strain curve, and a toughness value of the sample is calculated based on an integral area of a tensile stress-strain curve (MJ/m³); qualitative stress-strain curves in FIG. 26A show that a mechanical property strengthening effect of the (NH₄)₂SO₄ treatment for the E-Col gel film is significantly dependent on a concentration of (NH₄)₂SO₄, and the (NH₄)₂SO₄ treatment has a much smaller strengthening effect for an S-Col gel film than for the E-Col gel film; it is summarized in FIG. 26B that, after S-Col and E-Col networks both are strengthened by the 4 M (NH₄)₂SO₄ treatment, a Young's modulus of the E-Col film increases by 50 times and a Young's modulus of the S-Col film increases merely by 6 times; it can be seen from FIG. 26C that the 4 M (NH₄)₂SO₄ treatment increases the toughness of the E-Col film by 16 times, but this treatment makes basically no contribution to the toughness strengthening for the S-Col film; and the above results show that, after the (NH₄)₂SO₄ treatment, the toughness of the E-Col network is significantly improved compared with the toughness of the S-Col network, indicating that the collagen gel films with different assembled structures have different mechanical responses for a Hofmeister effect;

FIGS. 27A-27C show quantitative characterization results of mechanical properties of an E-Col film strengthened based on a Hofmeister effect, where a gel film is soaked in sodium carbonate (Na₂CO₃) solutions with different concentrations (1 M, 2 M, and 2.5 M) at room temperature for 12 h, and then an Electro-Force 3200 biodynamic tester is used to investigate the tensile properties of the gel film at room temperature, where a stretching rate is set at 10 mm/min to obtain a stress-strain curve of the gel film; stress-strain curves of the E-Col gel film treated with Na₂CO₃ of different concentrations in FIG. 27A show that Na₂CO₃ as a Hofmeister salt with a strong hydration ability can also strengthen the E-Col network, and a strengthening effect increases with the increase of a concentration of the salt; FIGS. 27B-27C show that the E-Col gel film is significantly strengthened after being treated with 2 M Na₂CO; for 24 h, but the strengthened E-Col gel film is gradually softened to an initial soft state after being treated in SBF for 24 h, that is, a strengthening process of the E-Col network by Na₂CO; is a reversible process, and the strengthened E-Col gel film returns to a soft state after Hofmeister salt ions are leached out, indicating that a mechanical strengthening effect based on a Hofmeister effect for the E-Col film is reversible;

FIGS. 28A-28C show test results of a mechanically-strengthened E-Col film used for artery banding in vivo, where 2-month-old New Zealand white rabbits are selected as experimental objects, and a heart and surgical sites are shown in FIG. 28A; a diameter of a pulmonary artery is reduced with the E-Col film strengthened by Na₂CO₃ as a surgical band, as shown in FIG. 28B; and color Doppler images of a heart in FIG. 28C show that the surgical band reduces the diameter of the pulmonary artery from a preoperative diameter Φ=0.63 cm to a postoperative diameter Φ₀=0.43 cm, with a reduction of about 68%, indicating that E-Col strengthened by Na₂CO₃ can provide a high mechanical strength to significantly reduce a diameter of a pulmonary artery; and the diameter of the pulmonary artery returns to 75% of the preoperative diameter of the pulmonary artery (Φ₁=0.48 cm) on day 1 after a surgery, and returns to the preoperative diameter of the pulmonary artery on day 3 after the surgery; and

FIGS. 29A-29C show blood flow velocities (VELs) and pressure gradients (PGs) of artery vessels before and after a surgery that are tested by Doppler ultrasonography, where a VEL decreases from 119 cm/s before the surgery to 93.1 cm/s after the surgery, and a PG significantly decreases from 6 mmHg before the surgery to 3 mmHg after the surgery, indicating that a strengthened E-Col band implanted around a pulmonary artery can significantly constrict the pulmonary artery to allow the short-term restriction of VEL and the reduction of a blood flow pressure.

### DETAILED DESCRIPTION OF THE EMBODIMENTS

### Example 1: A First Preparation Example of a Collagen Material

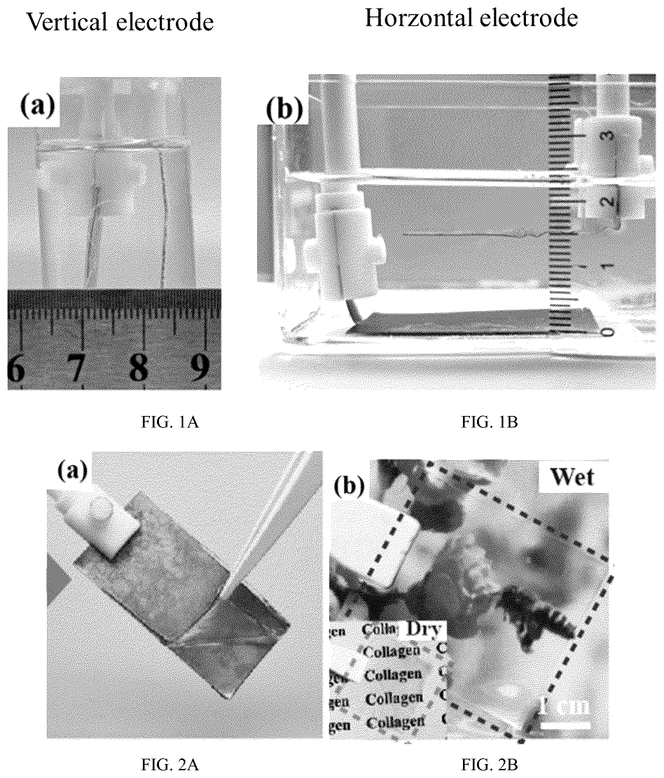

(1) Preparation of a first collagen solution: 400 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 3.5; and the solution was placed in a dialysis bag (Mw_{cut off}=7.0 kDa), the dialysis bag was placed in a beaker with 1,000 mL of water and 15 mL of glacial acetic acid, and dialysis was conducted at 4° C. for 72 d to remove small-molecule impurities to obtain a 10 mg/mL collagen-containing viscous solution.
(2) 80 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A titanium sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 1.5 cm between the electrodes; and the second collagen solution (concentration: 10 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 800 s at a constant current density of 6.67 mA/cm² and a voltage variation range of 0.22 V/cm² to 1.67 V/cm², where half reactions at the electrodes were as follows:

anode: 2H₂O−4e⁻→4H⁺O₂; and

cathode: 4H₂O+4e⁻→4OH⁻+2H₂.

At the end of the EDP, a collagen gel film appeared on the cathode, as shown in FIG. 2A. The cathode with the collagen gel film was rinsed multiple times with UPW, and then the collagen material E-Col was stripped from the cathode. When the electrodes are arranged either horizontally or vertically, a collagen material can be prepared. However, it is found that a material prepared with the electrodes arranged vertically will has a thin upper part and a thick lower part due to a gravity, which can be avoided when the electrodes are arranged horizontally. The E-Col collagen material has very uniform appearance and is highly-transparent in both dry and wet states, as shown in FIG. 2B.

### Example 2: A Second Preparation Example of a Collagen Material

(1) Preparation of a first collagen solution: 800 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 3.5; and the solution was placed in a dialysis bag (Mw_{cut off}=7.0 kDa), the dialysis bag was placed in a beaker with 1,000 mL of water and 15 mL of glacial acetic acid, and dialysis was conducted at 4° C. for 72 d to remove small-molecule impurities to obtain a 20 mg/mL collagen-containing viscous solution.
(2) 160 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A titanium sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 1.5 cm between the electrodes; and the second collagen solution (concentration: 20 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 800 s at a constant current density of 6.67 mA/cm² and a voltage variation range of 0.22 V/cm² to 1.67 V/cm²; and at the end of the EDP, a collagen gel film was successfully prepared on the cathode.

### Example 3: A Third Preparation Example of a Collagen Material

(1) Preparation of a first collagen solution: 40 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 3.5; and the solution was placed in a dialysis bag (Mw_{cut off}=7.0 kDa), the dialysis bag was placed in a beaker with 1,000 mL of water and 15 mL of glacial acetic acid, and dialysis was conducted at 4° C. for 72 d to remove small-molecule impurities to obtain a 1 mg/mL collagen-containing solution.
(2) 50 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A titanium sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 1.5 cm between the electrodes; and the second collagen solution (concentration: 1 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 800 s at a constant current density of 6.67 mA/cm² and a voltage variation range of 0.22 V/cm² to 1.67 V/cm²; and at the end of the EDP, a collagen gel film was successfully prepared on the cathode.

### Example 4: A Fourth Preparation Example of a Collagen Material

(1) Preparation of a first collagen solution: 400 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 2.0; and the solution was placed in a dialysis bag (Mw_{cut off}=7.0 kDa), the dialysis bag was placed in a beaker with 1,000 mL of water and 200 mL of glacial acetic acid, and dialysis was conducted at 4° C. for 72 d to remove small-molecule impurities to obtain a 10 mg/mL collagen-containing solution.
(2) 80 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A titanium sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 1.5 cm between the electrodes; and the second collagen solution (concentration: 10 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 800 s at a constant current density of 6.67 mA/cm² and a voltage variation range of 0.22 V/cm² to 1.67 V/cm²; and at the end of the EDP, a collagen gel film was successfully prepared on the cathode.

### Example 5: A Fifth Preparation Example of a Collagen Material

(1) Preparation of a first collagen solution: 400 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 4.0; and the solution was placed in a dialysis bag (Mw_{cut off}=7.0 kDa), the dialysis bag was placed in a beaker with 1,000 mL of water and 20 μL of glacial acetic acid, and dialysis was conducted at 4° C. for 72 d to remove small-molecule impurities to obtain a 10 mg/mL collagen-containing solution.
(2) 80 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A titanium sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 1.5 cm between the electrodes; and the second collagen solution (concentration: 10 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 800 s at a constant current density of 6.67 mA/cm² and a voltage variation range of 0.22 V/cm² to 1.67 V/cm²; and at the end of the EDP, a collagen gel film was successfully prepared on the cathode.

### Example 6: A Sixth Preparation Example of a Collagen Material

(1) Preparation of a first collagen solution: 400 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 3.5; and the solution was placed in a dialysis bag (Mw_{cut off}=7.0 kDa), the dialysis bag was placed in a beaker with 1,000 mL of water and 15 mL of glacial acetic acid, and dialysis was conducted at 4° C. for 72 d to remove small-molecule impurities to obtain a 10 mg/mL collagen-containing solution.
(2) 50 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A titanium sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 1.5 cm between the electrodes; and the second collagen solution (concentration: 10 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 800 s at a constant current density of 6.67 mA/cm² and a voltage variation range of 0.22 V/cm² to 1.67 V/cm²; and at the end of the EDP, a collagen gel film was successfully prepared on the cathode.

### Example 7: A Seventh Preparation Example of a Collagen Material

(1) Preparation of a first collagen solution: 400 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 3.5; and the solution was placed in a dialysis bag (Mw_{cut off}=7.0 kDa), the dialysis bag was placed in a beaker with 1,000 mL of water and 15 mL of glacial acetic acid, and dialysis was conducted at 4° C. for 72 d to remove small-molecule impurities to obtain a 10 mg/mL collagen-containing solution.
(2) 200 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A titanium sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 1.5 cm between the electrodes; and the second collagen solution (concentration: 10 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 800 s at a constant current density of 6.67 mA/cm² and a voltage variation range of 0.22 V/cm² to 1.67 V/cm²; and at the end of the EDP, a collagen gel film was successfully prepared on the cathode.

### Example 8: An Eighth Preparation Example of a Collagen Material

(1) Preparation of a first collagen solution: 400 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 3.5; and the solution was placed in a dialysis bag (Mw_{cut off}=7.0 kDa), the dialysis bag was placed in a beaker with 1,000 mL of water and 15 mL of glacial acetic acid, and dialysis was conducted at 4° C. for 72 d to remove small-molecule impurities to obtain a 10 mg/mL collagen-containing solution.
(2) 200 L/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A platinum sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 1.5 cm between the electrodes; and the second collagen solution (concentration: 10 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 800 s at a constant current density of 6.67 mA/cm² and a voltage variation range of 0.22 V/cm² to 1.67 V/cm²; and at the end of the EDP, a collagen gel film was successfully prepared on the cathode.

### Example 9: A Ninth Preparation Example of a Collagen Material

(1) Preparation of a first collagen solution: 400 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 3.5; and the solution was placed in a dialysis bag (Mw_{cut off}=7.0 kDa), the dialysis bag was placed in a beaker with 1,000 mL of water and 15 mL of glacial acetic acid, and dialysis was conducted at 4° C. for 72 d to remove small-molecule impurities to obtain a 10 mg/mL collagen-containing solution.
(2) 200 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A Pt sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 0.5 cm between the electrodes; and the second collagen solution (concentration: 10 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 800 s at a constant current density of 6.67 mA/cm² and a voltage variation range of 0.22 V/cm² to 1.67 V/cm²; and at the end of the EDP, a collagen gel film was successfully prepared on the cathode.

### Example 10: A Tenth Preparation Example of a Collagen Material

(1) Preparation of a first collagen solution: 400 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 3.5; and the solution was placed in a dialysis bag (Mw_{cut off}=7.0 kDa), the dialysis bag was placed in a beaker with 1,000 mL of water and 15 mL of glacial acetic acid, and dialysis was conducted at 4° C. for 72 d to remove small-molecule impurities to obtain a 10 mg/mL collagen-containing solution.
(2) 200 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A Pt sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 3.0 cm between the electrodes; and the second collagen solution (concentration: 10 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 800 s at a constant current density of 6.67 mA/cm² and a voltage variation range of 0.22 V/cm² to 1.67 V/cm²; and at the end of the EDP, a collagen gel film was successfully prepared on the cathode.

### Example 11: An Eleventh Preparation Example of a Collagen Material

(1) Preparation of a first collagen solution: 400 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 3.5; and the solution was placed in a dialysis bag (Mw_{cut off}=7.0 kDa), the dialysis bag was placed in a beaker with 1,000 mL of water and 15 mL of glacial acetic acid, and dialysis was conducted at 4° C. for 72 d to remove small-molecule impurities to obtain a 10 mg/mL collagen-containing solution.
(2) 200 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A Pt sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 3.0 cm between the electrodes; and the second collagen solution (concentration: 10 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 500 s at a constant current density of 6.67 mA/cm² and a voltage variation range of 0.22 V/cm² to 1.67 V/cm²; and at the end of the EDP, a collagen gel film was successfully prepared on the cathode.

### Example 12: A Twelfth Preparation Example of a Collagen Material

(1) Preparation of a first collagen solution: 400 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 3.5; and the solution was placed in a dialysis bag (Mw_{cut off}=7.0 kDa), the dialysis bag was placed in a beaker with 1,000 mL of water and 15 mL of glacial acetic acid, and dialysis was conducted at 4° C. for 72 d to remove small-molecule impurities to obtain a 10 mg/mL collagen-containing solution.
(2) 200 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide.
(3) A Pt sheet was adopted as a cathode (cathode size: 2 cm×3 cm), and a platinum wire or a platinum sheet was adopted as an anode; the two electrodes were horizontally arranged in parallel in an electrolytic cell (as shown in FIG. 1B) with a distance of 3.0 cm between the electrodes; and the second collagen solution (concentration: 10 mg/mL) obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI660E, a voltage was applied to the cathode, and EDP was conducted for 3000 s at a constant current density of 6.67 mA/cm² and a voltage variation range of 0.22 V/cm² to 1.67 V/cm²; and at the end of the EDP, a collagen gel film was successfully prepared on the cathode.

### Example 13: Characterization of Physical and Chemical Properties of a Collagen Material E-Col Prepared by an EDP Technology

E-Col was prepared by the same method as in Example 1, where gel E-Col with a thickness of about 400 μm was obtained by controlling a current intensity and an application time of the current intensity. For comparison, a collagen solution obtained after dialysis was also used to prepare a collagen film S-Col by a solution method, including: a pH of the acidic collagen solution (5 mg/mL; and pH=3.5) was adjusted with 0.5 M NaOH to 7.2, then a resulting collagen solution was poured into a round film Petri dish (a collagen content per unit area was the same as a mass of EDP-assembled collagen per unit area), and the Petri dish was incubated at 37° C. for 12 h to allow complete gelation; and a resulting gel was dehydrated for 48 h at room temperature to produce a milky-white translucent gel film with a thickness of about 400 μm.

### (1) Optical Property Test

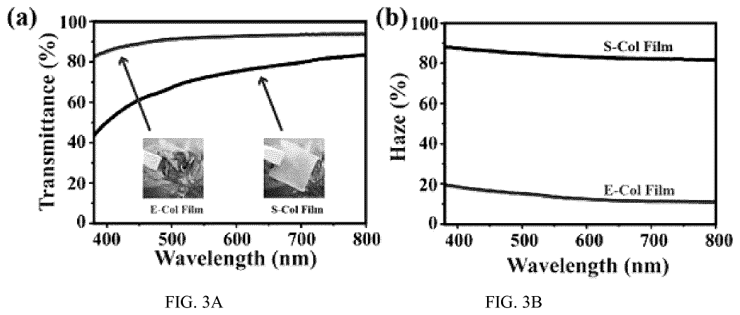

The prepared S-Col and E-Col gel films each were soaked in UPW for 1 h to allow a saturated water content, and then cut into square samples each with a fixed size; and each square sample was placed in a cuvette, and then the transmittance (%) and haze (%) of the square sample in a visible light wavelength range of 380 nm to 800 nm were detected by a UV-visible spectrophotometer (Lambda 950) (a background of the cuvette needed to be deducted from a detected value). The E-Col exhibited a high optical transparency, which was of nearly 90% in a wavelength range of 450 nm to 780 nm (visible light range); and the collagen gel film also had a very low haze, which was merely of 10% in the visible light range, as shown in FIGS. 3A-3B.

### (2) Micromorphological Characterization

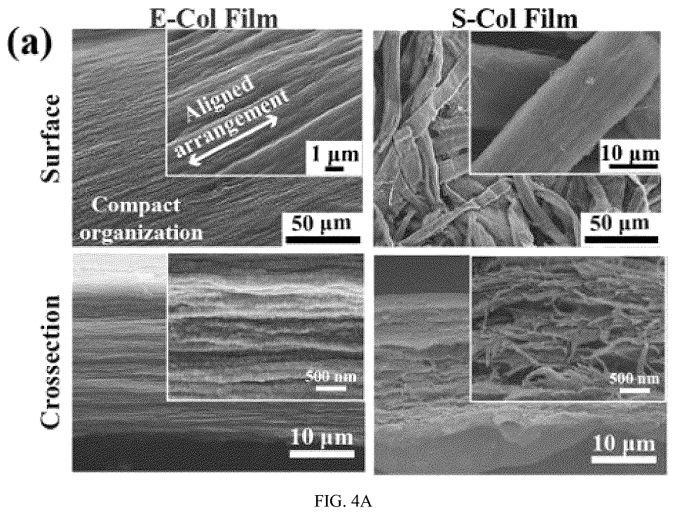

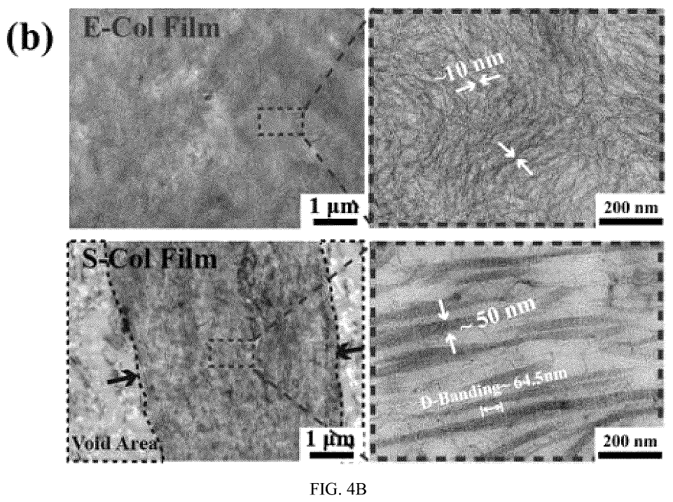

Microscopic morphology analysis was conducted by SEM (S-4800, Hitachi). An internal ultrastructure was observed by TEM (JEM-2100, JEOL). The prepared S-Col and E-Col gel film materials each were dehydrated with ethanol in different volume fractions, and then embedded into an Eponate12 resin (Ted Pella, Redding, CA). A 60 nm to 90 nm-thick sheet sample was placed on a bare copper grid, and then stained with uranyl acetate and lead. An image was acquired at an accelerating voltage of 200 kV. FIGS. 4A-4B show surface and cross-section SEM images of the collagen films prepared by the different methods. As shown in FIG. 4A, the E-Col film had a dense organizational structure (density: 0.88 g/cm³), and there was the oriented arrangement of nanofibers at both a surface and a cross section; and in contrast, the S-Col film assembled in a solution had a loose network inside (density: 0.45 g/cm³), and coarse fibers (diameter: about a few micrometers) were randomly aggregated in the film.

TEM images in FIG. 4B showed that the E-Col was obtained through tight arrangement of fine microfibers, and high-magnification TEM images revealed that the microfibers had a diameter of about 10 nm and did not have significant D-band characteristics of collagen fibers; and in contrast, there were loosely-arranged fibers at a micro-scale in the S-Col film, and high-magnification TEM images showed that micro-scale fibers in the S-Col film were fibrils with a diameter of 50 nm and the fibrils had significant D-band (about 64.5 nm) characteristics of type I collagen.

### (3) Orientation Characterization

An oriented structure of E-Col was investigated by POM and SAXS.

An SAXS experiment was conducted on a BL19U2 SAXS beam of the Shanghai Synchrotron Radiation Facility (SSRF). Scattering data were acquired by illuminating with an X-ray beam with a distance of 1,900 mm between a sample and a detector. A sample was measured for 60 s to obtain a scattering signal. 2D SAXS data were averaged into a one-dimensional (1D) scattering intensity curve and q, a background was set as a polynomial function, and a scattering minimum was calculated according to each SAXS curve. A Herman's orientation factor (f) was calculated according to formulas (I) and (II) to quantify an alignment degree of a collagen network:

$$fc = \frac{3 〈 {cos}^{2} ϕ 〉 - 1}{2} and \qquad formula ( I )$$

$$〈 {cos}^{2} ϕ 〉 = \frac{{\int }_{0}^{π / 2} I ( ϕ ) {cos}^{2} ϕsinϕ d ϕ}{{\int }_{0}^{π / 2} I ( ϕ ) sin ϕ d ϕ} , \qquad formula ( II )$$

where φ represents an azimuth angle, and I(ϕ) represents a 1D intensity distribution with an azimuth angle after a background intensity is deducted; average cos²ϕ is calculated by integrating an intensity of a particular 2θ diffraction peak along φ according to the above formula; and for an isotropic material, f_{c}=0, and for an anisotropic material, f_{c}=1.

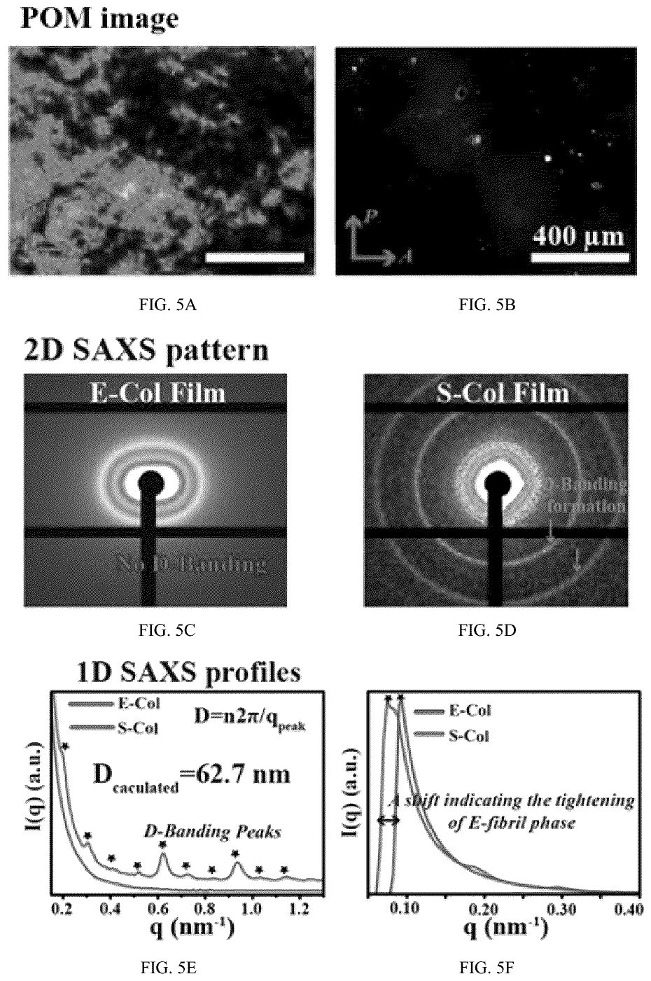

As shown by POM images in FIGS. 5A-5B, the S-Col film had no obvious optical birefringence phenomenon, which was manifested as an isotropic structure; and an optical birefringence phenomenon was observed in some regions of the E-Col gel film, indicating an oriented structure in these regions. As shown by SAXS data in FIGS. 5C-5D, a 2D SAXS pattern of the S-Col had a ring with an almost consistent intensity, indicating an isotropic structure; and a 2D SAXS pattern of the E-Col had a significantly-elongated ring, indicating an anisotropic structure. As shown by 1D-SAXS profiles in FIG. 5E, when a q value was in a range of 0.2 nm⁻¹ to 1.2 nm⁻¹, the S-Col had a significant D-band characteristic scattering peak in a 1D-SAXS profile (a D band calculated by the Bragg equation was at about 62.7 nm), and in contrast, the E-Col did not have a significant D-band characteristic peak, indicating an amorphous structure. As shown by 1D-SAXS profiles in FIG. 5F, when a q value was in a range of 0.05 nm⁻¹ to 0.4 nm⁻¹, the E-Col film shifted to a higher q value than the S-Col film, indicating the tightened arrangement of fibers.

### Example 14: Characterization of Dynamic and Static Mechanical Properties of a Collagen Material Prepared by an EDP Technology

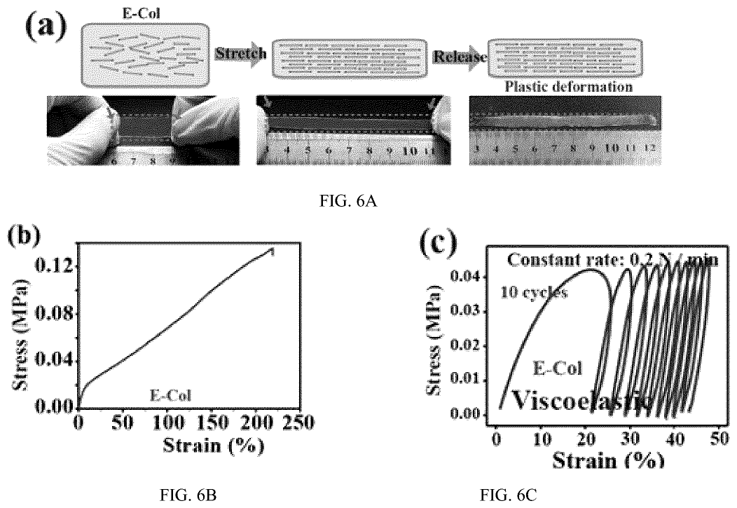

The collagen film in Example 1 was cut into rectangular sample strips each with a length of 30 mm and a width of 10 mm. After being stretched, the rectangular sample strips underwent a significant plastic deformation; and after a stretching force was removed, the rectangular sample strips did not return to their original shapes, as shown in FIG. 6A. An Electro-Force 3200 biodynamic tester was used to investigate the static and dynamic tensile properties of the collagen film at room temperature.

For a static mechanical test, a stretching rate was set at 10 mm/min to obtain a stress-strain curve of the collagen film. The E-Col gel film had a Young's modulus of 0.32±0.11 MPa (allowing a large deformation), an elongation at break of 220.41±5.07%, and a tensile strength of 0.13±0.03 MPa. The E-Col gel film underwent a stress yield in a very small region, indicating that there was merely a weak crosslinking mechanism inside (namely, a non-covalent bonding interaction), as shown in FIG. 6B.

For a dynamic mechanical test, loading and unloading processes of the E-Col film were set at 0.001 N to 0.04 N. A stretching rate was set at 0.2 N min 1, and 10 cycles were adopted to obtain a dynamic cyclic tensile curve of the collagen film. The E-Col film underwent a large deformation, and there was a significant lag between loading and unloading cycles, indicating viscoelasticity, as shown in FIG. 6C.

### Example 15: Reversibility of a Collagen Material Prepared by an EDP Technology

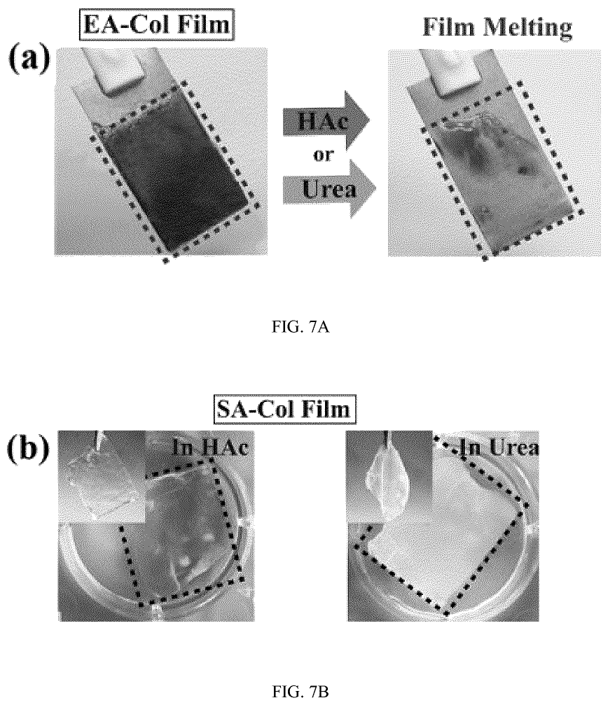

When the collagen film prepared in Example 1 was soaked in a 0.1 M acetic acid solution with a pH of 3.5 or a 0.1 M urea (a strong hydrogen bond-shielding agent) solution, the collagen film E-Col was rapidly dissolved within less than 10 min, as shown in FIGS. 7A-7B. In contrast, the S-Col remained stable persistently. It indicated that the intermolecular binding in the E-Col film mainly depended on some weak molecular interactions, such as hydrogen bonding and hydrophobic interactions. A solution of E-Col dissolved by acetic acid could undergo EDP once again to obtain an E-Col material.

### Example 16: Controllable Preparation of an E-Col Film

Based on the preparation method of Example 1, collagen films with different thicknesses were prepared with a size of the cathode titanium sheet reduced to 1 cm×1 cm and the constant current density (2.5 mA/cm², 5 mA/cm², and 10 mA/cm²) and deposition time (500 s, 1,000 s, 2,000 s, and 3,000s) changed.

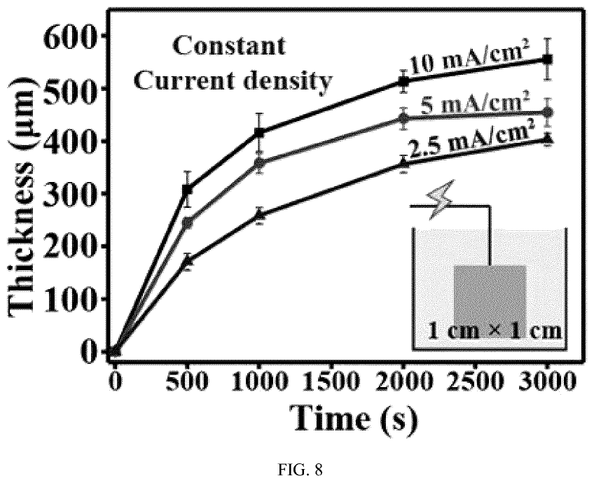

Thicknesses of samples in a wet state could be measured to obtain relationships of the thicknesses with the constant current density and deposition time. As shown in FIG. 8, a thickness of a collagen film could be controlled by changing the constant current density and deposition time.

When the deposition time varied from 0 s to 3,000 s, the thickness of the collagen film could vary from 0 μm to 400 μm at a current density of 2.5 mA/cm²;
the thickness of the collagen film could vary from 0 μm to 450 μm at a current density of 5 mA/cm²; and
the thickness of the collagen film could vary from 0 μm to 550 μm at a current density of 10 mA/cm².

### Example 17: Preparation of Collagen Materials with Different Macroscopic Geometric Shapes

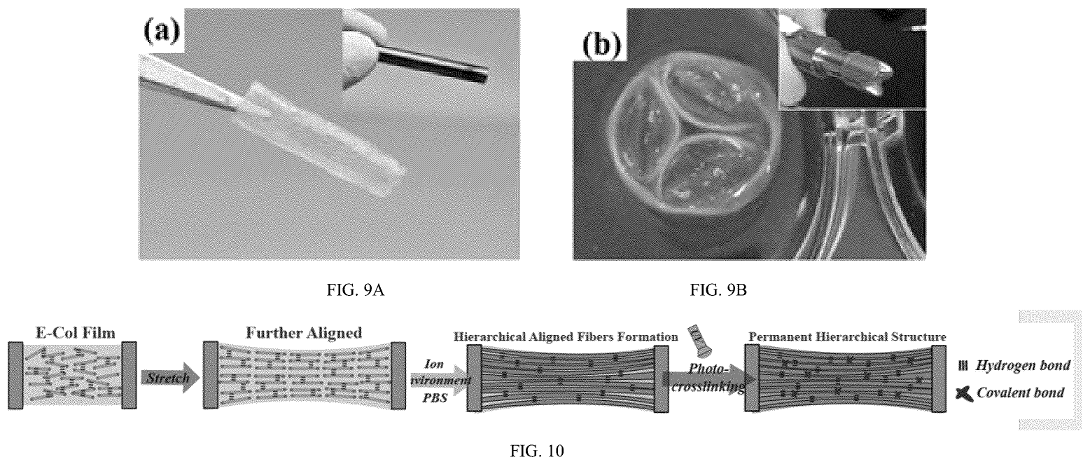

To demonstrate the versatility of the improved EDP technology, macroscopic collagen structures of various shapes were prepared. A shape of a cathode was changed, or a titanium tube (outer diameter: 6.0 mm, and inner diameter: 5.0 mm) or a stainless steel heteromorphic column with a valve-shaped end was used as a cathode to prepare various heteromorphic structural materials by the EDP technology in Example 1. When the cathode was a titanium tube, a hollow collagen tube could be obtained, as shown in FIG. 9A. When a shape of the cathode was similar to a shape of a heart valve, a heart valve-like heteromorphic collagen material could be obtained, as shown in FIG. 9B.

### Example 18: A First Preparation Example of a Collagen Film with a Long Range-Oriented and Crystalline Collagen Fiber Structure

### (1) Mechanical Stretching

The collagen film E-Col prepared according to the method in Example 1 was cut into rectangular sample strips each with a length of 30 mm and a width of 10 mm. A plurality of rectangular sample strips E-Col were soaked in UPW for 5 min and then stretched by an Electro-Force 3200 biodynamic tester in a length direction of the collagen film to a strain degree of 200%, such that microfibers inside the collagen film were further oriented in a forced direction to produce a long range-oriented collagen material. Finally, the stretched E-Col was soaked in ethanol to temporarily fix an oriented structure.

### (2) Ion Incubation

The long range-oriented collagen material obtained in the step (1) was subjected to ion incubation as follows: Two ends of a strip collagen film material were fixed by a tape in a Petri dish to make the strip collagen film material undergo a continuous external action force without shrinking, then 0.1 M PBS was added to the Petri dish, and the Petri dish was incubated for 24 h at room temperature to induce the re-arrangement of microfiber structures inside to produce crystalline large-diameter collagen fibers with D-band characteristics.

### (3) Chemical Crosslinking

Photocrosslinking: The highly-oriented and crystalline collagen film obtained in the step 2 was soaked in a 1 mg/mL riboflavin solution (90% v/v ethanol-water), and crosslinking was conducted for 24 h under 365 nm UV irradiation to further strengthen the mechanical properties of the material.

FIG. 10 is a schematic diagram of mechanical stretching, ion incubation, and chemical crosslinking of E-Col.

### Example 19: A Second Preparation Example of a Collagen Film with a Highly-Oriented and Crystalline Collagen Fiber Structure

### (1) Mechanical Stretching

The collagen film E-Col prepared according to the method in Example 1 was cut into rectangular sample strips each with a length of 20 mm and a width of 20 mm. A plurality of rectangular sample strips E-Col were soaked in UPW for 10 min and then stretched by an Electro-Force 3200 biodynamic tester in a length direction of the collagen film to a strain degree of 50%, such that microfibers inside the collagen film were further oriented in a forced direction to produce a long range-oriented collagen material. Then the stretched E-Col was soaked in ethanol to temporarily fix an oriented structure.

### (2) Ion Incubation

The highly-oriented collagen material obtained in the step (1) was subjected to ion incubation as follows: Two ends of a strip collagen film material were fixed by a tape in a Petri dish to make the strip collagen film material undergo a continuous external action force without shrinking, then 0.05 M PBS was added to the Petri dish, and the Petri dish was incubated for 30 h at room temperature to induce the re-arrangement of microfiber structures inside to produce crystalline large-diameter collagen fibers with D-band characteristics.

### (3) Chemical Crosslinking

Glutaraldehyde crosslinking: A glutaraldehyde solution (0.5% w/v, 90% v/v ethanol-water) was prepared, and the highly-oriented and crystalline collagen film obtained in the step (2) was soaked in the glutaraldehyde solution to allow crosslinking for 30 min. Then the collagen film was repeatedly rinsed with UPW to remove the residual glutaraldehyde component in the collagen film to obtain the collagen film with a highly-oriented and crystalline collagen fiber structure.

### Example 20: A Third Preparation Example of a Collagen Film with a Highly-Oriented and Crystalline Collagen Fiber Structure

### (1) Mechanical Stretching

The collagen film E-Col prepared according to the method in Example 1 was cut into rectangular sample strips each with a length of 20 mm and a width of 10 mm. A plurality of rectangular sample strips E-Col were soaked in UPW for 3 min and then stretched by an Electro-Force 3200 biodynamic tester in a length direction of the collagen film to a strain degree of 100%, such that microfibers inside the collagen film were further oriented in a forced direction to produce a highly-oriented collagen material. Finally, the stretched E-Col was soaked in ethanol to temporarily fix an oriented structure.

### (2) Ion Incubation

The highly-oriented collagen material obtained in the step (1) was subjected to ion incubation as follows: Two ends of a strip collagen film material were fixed by a tape in a Petri dish to make the strip collagen film material undergo a continuous external action force without shrinking, then 0.2 M PBS was added to the Petri dish, and the Petri dish was incubated for 20 h at room temperature to induce the re-arrangement of microfiber structures inside to produce crystalline large-diameter collagen fibers with D-band characteristics.

### (3) Chemical Crosslinking

Genipin crosslinking: A 1% genipin solution was prepared, and the highly-oriented and crystalline collagen film obtained in the step (2) was soaked in the genipin solution to allow crosslinking for 10 h. Then the collagen film was repeatedly rinsed with UPW to remove the residual genipin component in the collagen film to obtain the long range-oriented collagen film.

### Example 21: A Fourth Preparation Example of a Collagen Film with a Highly-Oriented and Crystalline Collagen Fiber Structure

### (1) Mechanical Stretching

The collagen film E-Col prepared according to the method in Example 1 was cut into rectangular sample strips each with a length of 20 mm and a width of 20 mm. A plurality of rectangular sample strips E-Col were soaked in UPW for 10 min and then stretched by an Electro-Force 3200 biodynamic tester in a length direction of the collagen film to a strain degree of 50%, such that microfibers inside the collagen film were further oriented in a forced direction to produce a long range-oriented collagen material. Then the stretched E-Col was soaked in ethanol to temporarily fix an oriented structure.
(2) Ion incubation: The highly-oriented collagen material (50% stretched) obtained in the step (1) was subjected to ion incubation as follows: Two ends of a strip collagen film material were fixed by a tape in a Petri dish to make the strip collagen film material undergo a continuous external action force without shrinking, then 0.05 M PBS was added to the Petri dish, and the Petri dish was incubated for 18 h at 37° C. to induce the re-arrangement of microfiber structures inside to produce crystalline large-diameter collagen fibers with D-band characteristics.
(3) Chemical crosslinking: PAC crosslinking: A 1.5% PAC aqueous solution was prepared, and a pH of the PAC aqueous solution was adjusted with NaOH to 8.5. The highly-oriented and crystalline collagen film obtained in the step (2) was soaked in the PAC aqueous solution to allow crosslinking for 12 h. Then the collagen film was repeatedly rinsed with UPW to remove the residual polyphenol component in the collagen film to obtain the long range-oriented collagen film.

### Example 22: A Fifth Preparation Example of a Collagen Film with a Highly-Oriented and Crystalline Collagen Fiber Structure

The mechanical stretching and ion incubation in this example were the same as in Example 20. Photocrosslinking was adopted as the chemical crosslinking, and was specifically as follows:

The highly-oriented and crystalline collagen film obtained in the step (2) was soaked in a 0.5 mg/mL riboflavin solution (90% v/v ethanol-water), and crosslinking was conducted for 40 h under 365 nm UV irradiation to further strengthen the mechanical properties of the material.

### Example 23: A Sixth Preparation Example of a Collagen Film with a Highly-Oriented and Crystalline Collagen Fiber Structure

The ion incubation and chemical crosslinking were the same as in Example 18. Steps of the mechanical stretching were as follows:

The short range-oriented collagen film E-Col prepared according to the method in Example 1 was cut into rectangular sample strips each with a length of 40 mm and a width of 20 mm. A plurality of rectangular sample strips E-Col were soaked in UPW for 8 min and then stretched by an Electro-Force 3200 biodynamic tester in a length direction of the collagen film to a strain degree of 150%, such that microfibers inside the collagen film were further oriented in a forced direction to produce a long range-oriented collagen material. Finally, the stretched E-Col was soaked in ethanol for 10 min or more to temporarily fix an oriented structure.

### Example 24: Orientation Characterization of E-Col after Mechanical Stretching and Photocrosslinking

An internal oriented structure of mechanically-stretched E-Col prepared by the preparation method in Example 18 was investigated by POM (Nikon Eclipse Ci-L), 2DSAXS (BL19U2), and TEM (JEM-2100, JEOL).

A collagen film S-Col was prepared as a control sample by a solution method. A preparation method of the collagen film S-Col was as follows: a pH of the acidic collagen solution (5 mg/ml; and pH=3.5) was adjusted with 0.5 M NaOH to 7.2, then a resulting collagen solution was poured into a round film Petri dish (a collagen content per unit area was the same as a mass of EDP-assembled collagen per unit area), and the Petri dish was incubated at 37° C. for 12 h to allow complete gelation; and a resulting gel was dehydrated for 48 h at room temperature to produce a milky-white translucent gel film.

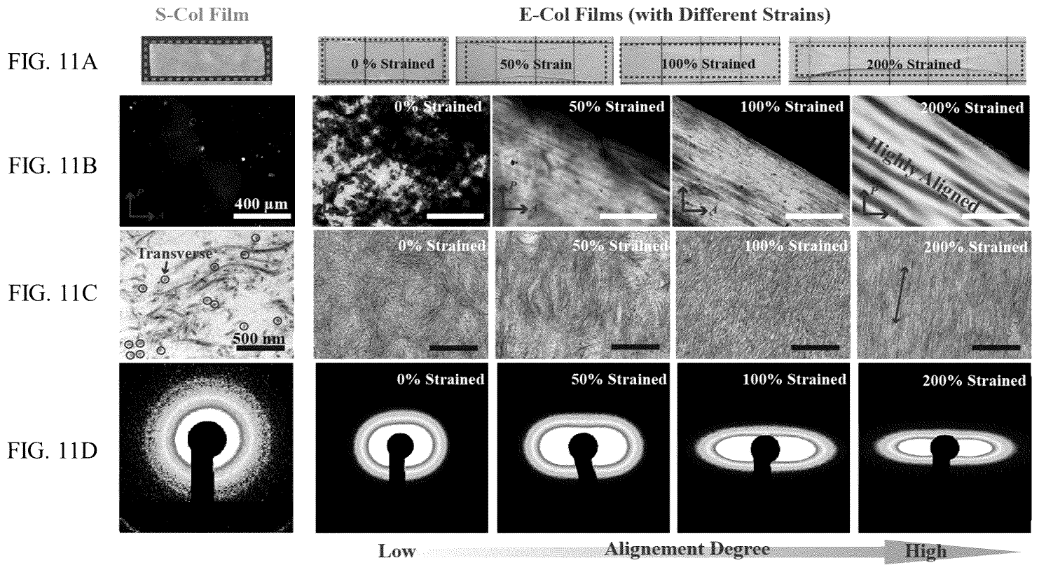

The appearance of each film was shown in FIG. 11A. As shown by observation results of POM in FIG. 11B, an S-Col film in a control group had no obvious optical birefringence phenomenon, which was manifested as an isotropic structure; there was an optical birefringence phenomenon in some regions of the E-Col film that was not stretched and deformed, indicating an ordered structure in these regions; and when the E-Col film was stretched to a high strain degree, an obvious optical birefringence phenomenon could be observed throughout the entire region of the E-Col film, and when a deformation degree was further increased to 200%, there was a bright birefringence color, indicating the formation of a highly-oriented structure in the E-Col film.

As shown by TEM images in FIG. 11C, the S-Col film in the control group had a loose isotropic structure (red circles indicate fibrils perpendicular to a cross section); a density and an orientation degree of the E-Col film could be significantly improved through mechanical stretching; and the greater the deformation induced by mechanical stretching, the denser the arrangement of internal microfibers and the higher the orientation degree.

As shown by 2D SAXS patterns in FIG. 11D, a 2D SAXS pattern of the S-Col film in the control group had a ring with an almost consistent intensity, which was consistent with an internal isotropic structure; and a 2D SAXS pattern of the E-Col film had a significantly-elongated ring, indicating the presence of an anisotropic nanofiber structure.

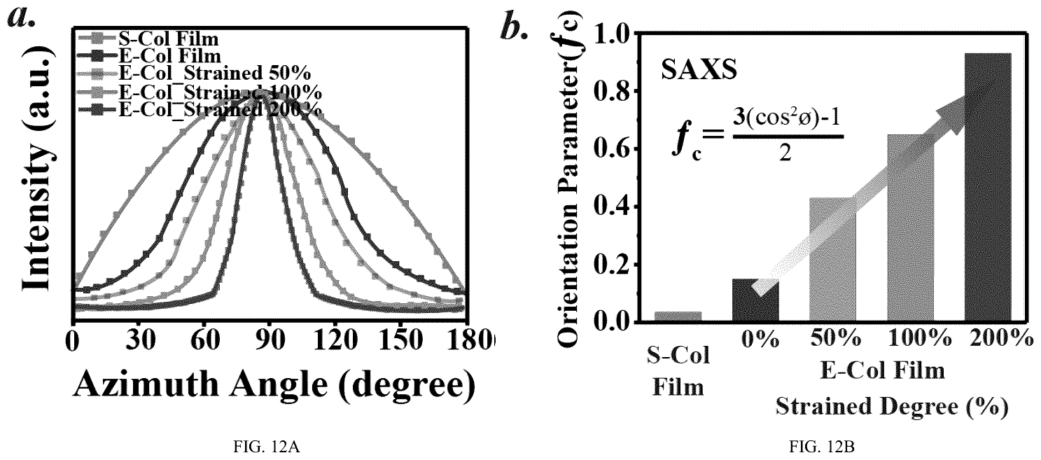

In order to quantitatively describe an orientation degree of the collagen film, azimuthal-integrated intensity distribution curves were further analyzed and plotted, as shown in FIG. 12A. The results showed that an azimuthal-integrated intensity distribution curve of the E-Col film gradually narrowed with the increase of a strain degree. A Herman's orientation parameter (f_{c}) was a quantitative index describing an orientation degree and could be calculated according to an azimuthal-integrated intensity distribution curve. As shown in FIG. 12B, an orientation degree of the S-Col film in the control group is almost 0 (f_{c}=0.02), and the E-Col film that was not stretched had a low orientation degree (f_{c}=0.15); and with the increase of a tensile strain of the E-Col film, the Herman's orientation parameter gradually increased from f_{c}=0.15 to f_{c}=0.93 (when a strain was 200%), indicating that the stretching of the E-Col film induced the formation of an oriented structure in a strain direction.

### Example 25: Morphological Characterization of E-Col after Mechanical Stretching, Ion Incubation, and Photocrosslinking

The E-Col was subjected to mechanical stretching (200% stretching) and ion incubation according to the method in Example 18, and then compared with a native tendon in terms of macroscopic and microscopic structures. Morphological data were acquired by SEM.

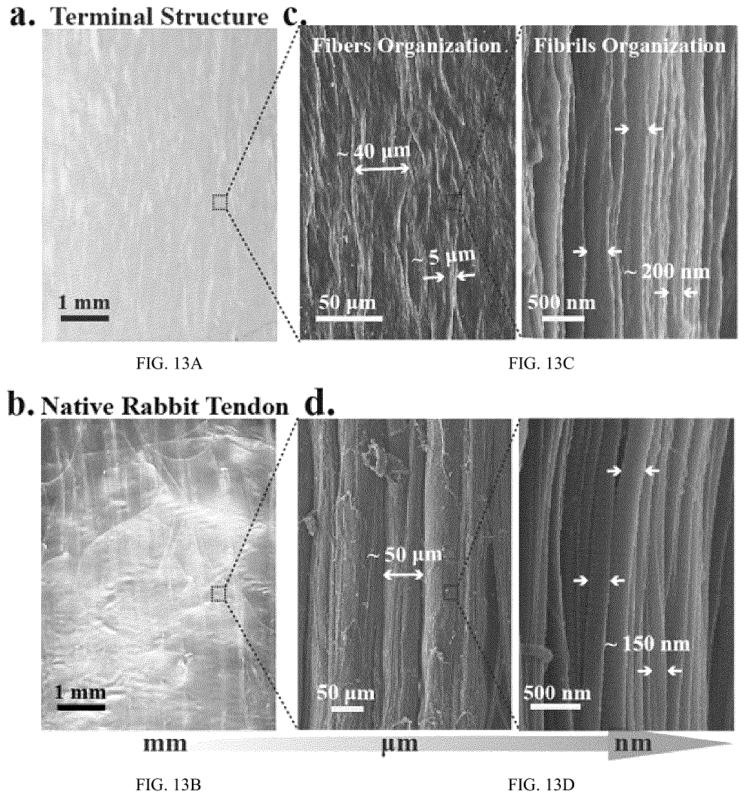

As shown in FIG. 13A, after the mechanical stretching and ion incubation, the E-Col film that was highly-transparent initially became milky-white and opaque and had millimeter-scale-oriented stripes on its surface, that is, the E-Col film became similar to the native tendon (FIG. 13B), which may be attributed to a change of an optical transparency caused by the formation of higher-order hierarchical structures (namely, large-diameter fibers). As shown by low-magnification and high-magnification SEM images in FIGS. 13C-13D, after PBS incubation, the E-Col film presented higher-order hierarchical structures, that is, densely-arranged fibers with a diameter of 5 μm to 10 μm.

### Example 26: Characterization of an Orientation Degree and a Crystal Form of E-Col after Mechanical Stretching and Ion Incubation

The E-Col was subjected to mechanical stretching (200% stretching), ion incubation, and photocrosslinking according to the method in Example 18, and then compared with a native tendon in terms of the orientation degree and crystal form.

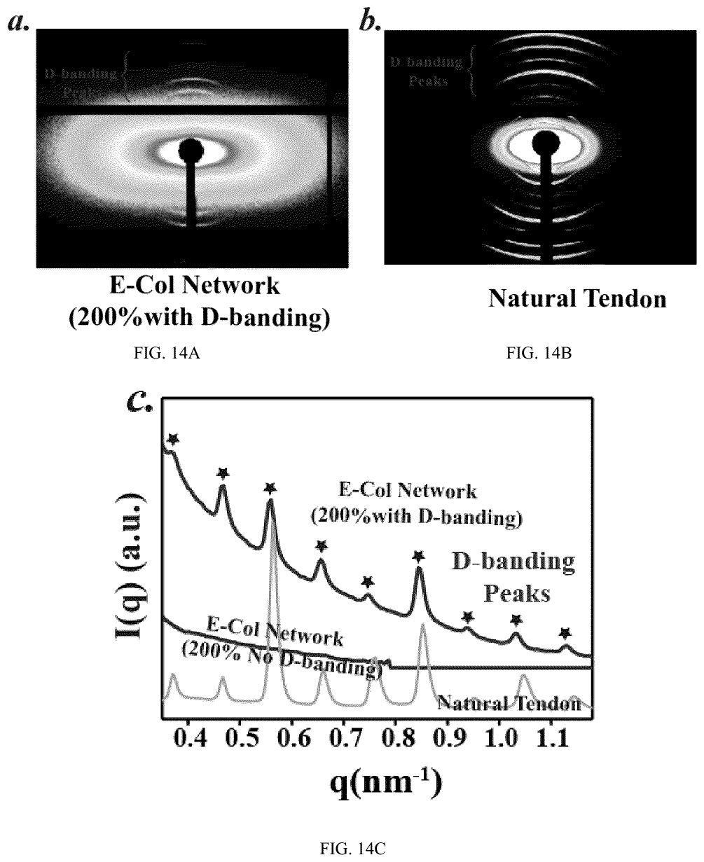

As shown by a 2D SAXS pattern in FIG. 14A, after 200% pre-stretching and ion incubation, the E-Col film still had a significantly-elongated ring, indicating that an anisotropic structure still retained after the ion incubation, and f_{c} was calculated to be about 0.52 to 0.53; and there was a significant D-band diffraction ring in the 2D SAXS pattern, indicating that collagen molecules were orderly arranged after the ion incubation. A 2D SAXS pattern of the native tendon in FIG. 14B also had a significant D-band diffraction ring, and f_{c} was calculated to be about 0.69 to 0.72. As shown by IDSAXS profiles in FIG. 14C, after the ion incubation, a similar crystal structure to the native tendon was produced in the E-Col film.

### Example 27: Characterization of Static Mechanical Properties of E-Col after Mechanical Stretching, Ion Incubation, and Photocrosslinking

The collagen film in Example 18 was subjected to 200% mechanical stretching, ion incubation, and photocrosslinking, and then compared with a native tendon in terms of mechanical properties. An Electro-Force 3200 biodynamic tester was used to investigate the static tensile properties of the collagen film and the native tendon at room temperature.

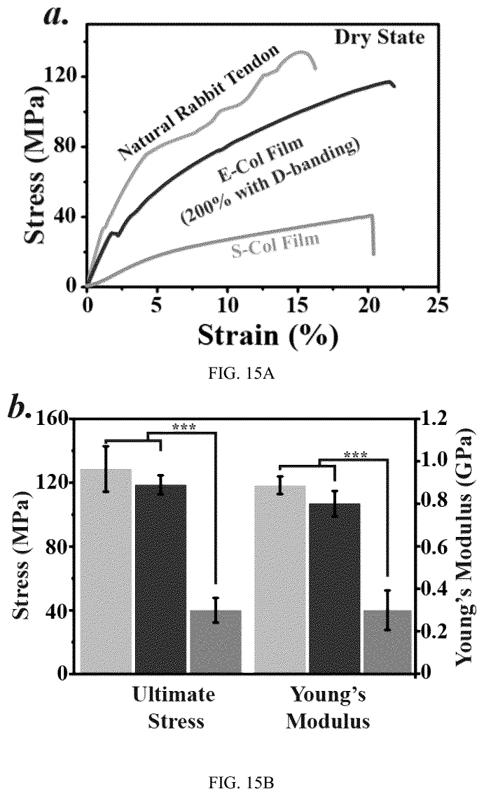

In order to investigate the contribution of a structure of a material to mechanical properties of the material, the material was tested in a dry state. A breaking stress and a Young's modulus of the material were shown in FIGS. 15A-15B, respectively. The E-Col material had a breaking stress of about 108±6 MPa, which was slightly lower than a breaking stress of the native tendon (128±14 MPa); and the E-Col material had a Young's modulus of 0.795±0.060 GPa, which basically reached a level (0.890±0.118 GPa) of the native tendon. It can be speculated that, because oriented structures and crystallization characteristics highly similar to those of the native tendon are produced after the mechanical stretching and ion incubation, the E-Col material exhibits excellent mechanical properties similar to those of the native tendon, which can provide a potential biomaterial for tendon/ligament repair.

### Example 28: A First Collagen Material Assembled by the EDP Technology

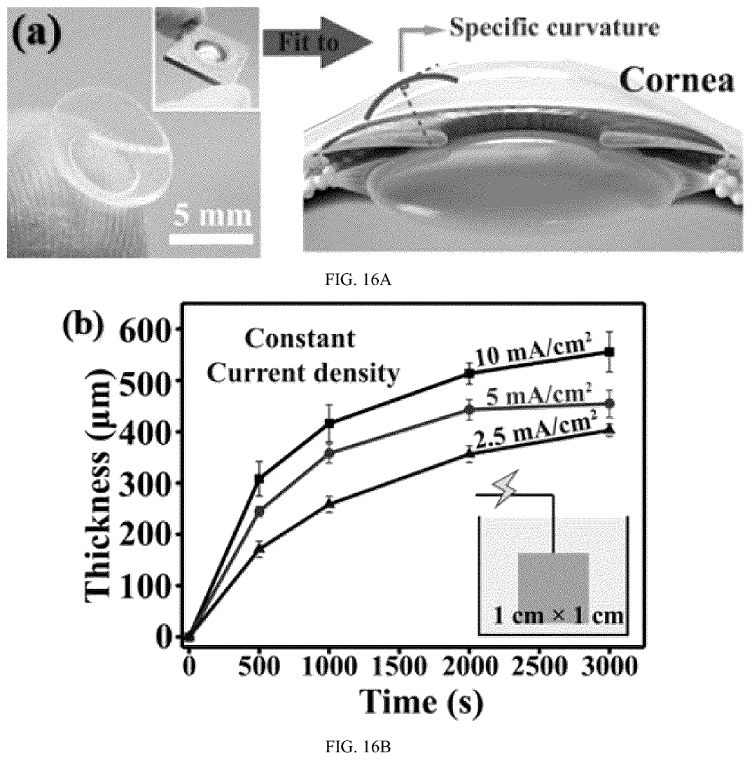

(1) Preparation of a first collagen solution: 400 mg of type I collagen was accurately weighed and dissolved in 40 mL of UPW, and then glacial acetic acid was added dropwise; a resulting system was thoroughly stirred to promote the complete dissolution of the type I collagen, and a pH of a final solution was adjusted to 3.5; and the solution was placed in a dialysis bag (Mw_{cut off}=7.0 kDa), the dialysis bag was placed in a beaker with 1,000 mL of water and 15 mL of glacial acetic acid, and dialysis was conducted at 4° C. for 72 d to remove small-molecule impurities to obtain a 10 mg/mL collagen-containing viscous solution (a mass of the collagen raw material added could be changed to make a collagen concentration of a final collagen solution vary in a range of 1 mg/mL to 20 mg/mL; when a collagen concentration was higher than 20 mg/mL, a resulting collagen solution lost fluidity; and a collagen concentration of 10 mg/mL was preferred).
(2) 100 μL/mL hydrogen peroxide was added to the first collagen solution obtained in the step (1), a resulting mixture was thoroughly stirred and then centrifuged at 8,000 rpm/min and 4° C. to remove bubbles, and a second collagen solution obtained after the centrifugation was stored in an ice-water mixed bath to prevent the decomposition of hydrogen peroxide (a concentration of the hydrogen peroxide added could be controlled in a range of 5 μl/mL to 200 μl/mL; and when a hydrogen peroxide concentration was higher than 200 μL/mL, hydrogen peroxide was easily decomposed directly in an electrolyte to produce bubbles).
(3) A titanium plate electrode with a fixed curvature (the curvature was 8.0, and could be adjusted in a range of 7.8 to 8.5 according to actual needs, as shown in FIG. 16A) was adopted as a working electrode, and a platinum wire or a platinum sheet (anode) was adopted as a counter electrode. The two electrodes were arranged in the following two ways: 1. The two electrodes were vertically arranged in parallel in an electrolytic cell. 2. The two electrodes were horizontally arranged in parallel in an electrolytic cell. A distance between the two electrodes was controlled at 1.5 cm. The second collagen solution with a concentration of 10 mg/mL obtained in the step (2) was carefully added to the electrolytic cell, where the addition of the second collagen solution should be slow to prevent bubbles caused by a too-high viscosity of a solution.
(4) Then the electrodes were connected to an electrochemical workstation CHI 660E, a voltage was applied to the cathode, and EDP was conducted for 1,500 s at a constant current density of 5 mA/cm² and a voltage variation range of 0.22 V/cm² to 1.67 V/cm², where half reactions at the electrodes were as follows:

anode: 2H₂O−4e⁻→4H⁺O₂; and

cathode: 4H₂O+4e⁻→4OH⁻+2H₂.

The current density and the deposition time could be controlled to obtain a collagen gel film with a specified thickness on the cathode (as shown in FIG. 16B). The working electrode with the collagen gel film was rinsed multiple times with UPW, and then the collagen material E-Col was stripped from the cathode. When the electrodes are arranged either horizontally or vertically, a collagen material can be prepared. However, it is found that a material prepared with the electrodes arranged vertically will has a thin upper part and a thick lower part due to a gravity, which can be avoided when the electrodes are arranged horizontally.

### Example 29: Chemical Crosslinking of a Collagen Material Prepared by an EDP Technology

A collagen film E-Col with a fixed curvature was prepared according to the method in Example 28. Conventional chemical crosslinking methods such as photocrosslinking and glutaraldehyde crosslinking could be adopted. The two chemical crosslinking methods were briefly described below:

Photocrosslinking: The collagen film prepared in Example 1 was soaked in a 1 mg/mL riboflavin solution (90% v/v ethanol-water), and crosslinking was conducted for 24 h under 365 nm UV irradiation to further strengthen the mechanical properties of the material.

Glutaraldehyde crosslinking: A glutaraldehyde solution (0.5% w/v, 90% v/v ethanol-water) was prepared, and then the collagen film prepared in Example 1 was soaked in the glutaraldehyde solution to allow crosslinking for 30 min; and then the collagen film was repeatedly rinsed with UPW to remove the residual glutaraldehyde component in the collagen film.

### Example 30: Characterization of Optical Properties of a Collagen Material Prepared by an EDP Technology after Chemical Crosslinking

A collagen film E-Col with a fixed curvature was prepared by the same method as in Example 28, where gel E-Col with a thickness of about 400 μm was obtained by controlling a current intensity and an application time of the current intensity. For comparison, a collagen solution obtained after dialysis was also used to prepare a collagen film S-Col by a solution method, including: a pH of the acidic collagen solution (5 mg/mL; and pH=3.5) was adjusted with 0.5 M NaOH to 7.2, then a resulting collagen solution was poured into a round film Petri dish (a collagen content per unit area was the same as a mass of EDP-assembled collagen per unit area), and the Petri dish was incubated at 37° C. for 12 h to allow complete gelation; and a resulting gel was dehydrated for 48 h at room temperature to produce a milky-white translucent gel film with a thickness of about 400 μm. The collagen film E-Col was subjected to photocrosslinking and glutaraldehyde crosslinking separately, and resulting collagen films were denoted as E-Col-UV and E-Col-GA, respectively; and in a blank group, the collagen film E-Col was not treated.

The prepared S-Col, E-Col-UV, E-Col-GA, and blank E-Col each were soaked in UPW for 1 h to allow a saturated water content, and then cut into square samples each with a fixed size; and each square sample was placed in a cuvette, and then the transmittance (%) and haze (%) of the square sample in a visible light wavelength range of 380 nm to 800 nm were detected by a UV-visible spectrophotometer (Lambda 950) (a background of the cuvette needed to be deducted from a detected value).

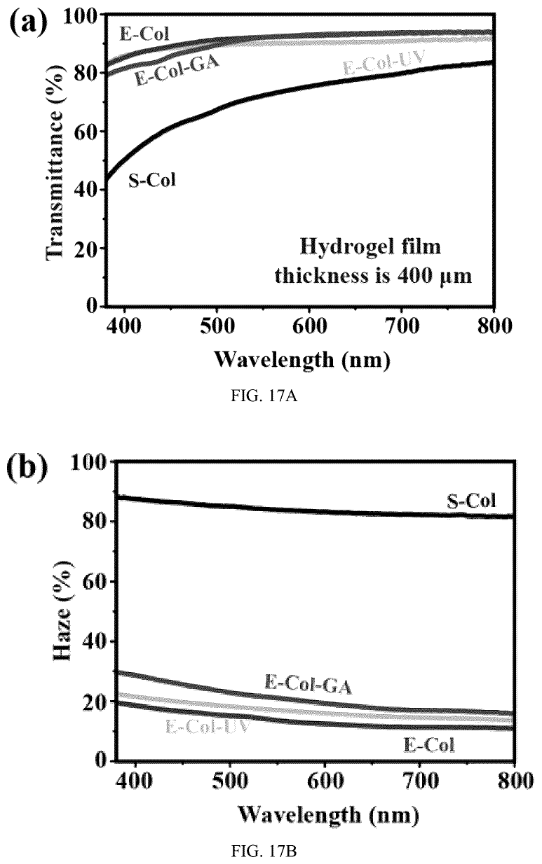

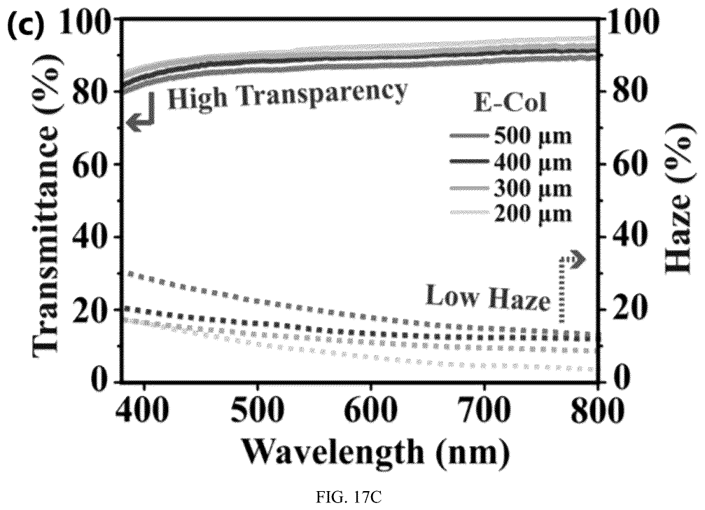

A transmittance of a collagen gel film material in each group increased with the increase of a wavelength in a range of 380 nm to 800 nm; and when the E-Col gel material was crosslinked by the different methods, an optical transparency of the E-Col gel material basically did not change, which was significantly better than an S-Col gel material assembled in a solution. A transmittance of a healthy human cornea at 430 nm was about 80%, and a transmittance of the healthy human cornea at 500 nm or more could be close to 100%. A transmittance of the E-Col gel material exceeded 80% at 400 nm before and after crosslinking, and with the increase of the wavelength, the transmittance was stabilized at about 94% when the wavelength was 500 nm or more, which was close to a transmittance level of the healthy human cornea. In addition, hazes of all samples decreased with the increase of a wavelength; and after crosslinking by different methods, the haze increased to some extent, but was basically maintained at a low level (lower than 30%), as shown in FIGS. 17A-17B.

A collagen film E-Col with a fixed curvature was prepared by the same method as in Example 28, where E-Col gel films with thicknesses of about 200 μm, 300 μm, 400 μm, and 500 μm were obtained by controlling a current intensity and an application time of the current intensity. Transmittances and hazes of these gel films were evaluated by the same method. As shown in FIG. 17C, the increase of a thickness basically did not affect the high transmittance and low haze of the material.

### Example 31: Micromorphological Characterization of a Collagen Material Prepared by an EDP Technology after Chemical Crosslinking

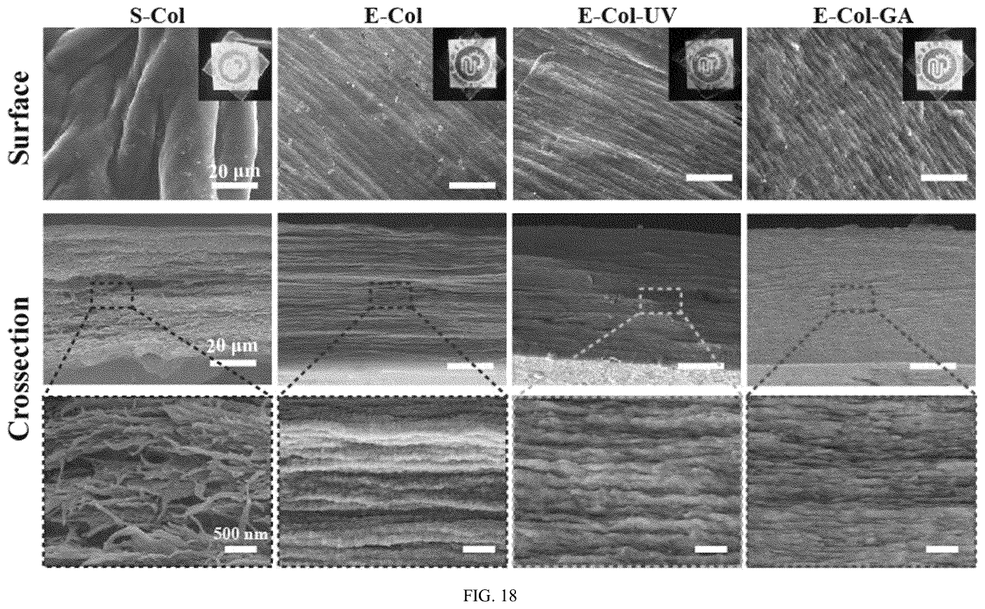

The S-Col, E-Col-UV, E-Col-GA, and E-Col collagen films prepared in Example 30 each were lyophilized, and then microscopic morphologies of the lyophilized collagen films were analyzed by SEM (S-4800, Hitachi), as shown in FIG. 18.

The S-Col film was milky-white and translucent, and had coarse fibrous structures on its surface and a morphology of loosely-stacked fibers at a cross section. The E-Col gel materials were highly-transparent; a surface morphology of an E-Col gel material showed the oriented arrangement of small-size fibers, and a cross-section morphology of an E-Col gel material showed the tight stacking of layered structures. After crosslinking, a surface of the material could still retain an oriented structure well, and it can be seen from a cross-sectional structure that the crosslinking increased the structural compactness to some extent. Structural observation results further proved that, because E-Col could retain an excellent microscopic morphology after crosslinking, E-Col also exhibited excellent optical properties at a macroscopic level.

### Example 32: Characterization of Mechanical Properties of a Collagen Material Prepared by an EDP Technology after Chemical Crosslinking

E-Col-UV, E-Col-GA, and E-Col collagen films were prepared by the same method as in Example 30 and cut into rectangular sample strips each with a length of 30 mm and a width of 10 mm, and an Electro-Force 3200 biodynamic tester was used to investigate mechanical properties of the collagen films at room temperature.

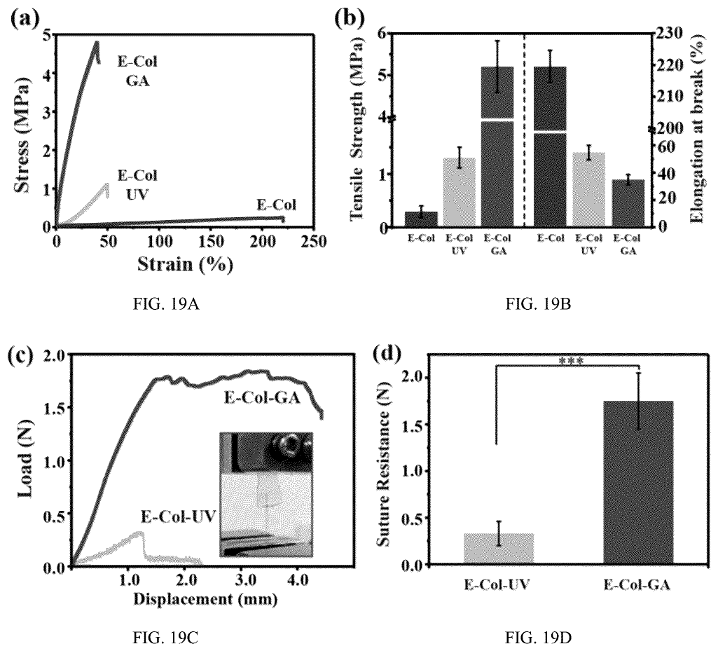

The tensile properties of the E-Col, E-Col-UV, and E-Col-GA gel film materials were first tested and compared. A stretching rate was set at 10 mm/min to obtain a stress-strain curve of the collagen film. As shown in FIGS. 19A-19B, compared with the E-Col gel film, the E-Col-UV and E-Col-GA crosslinked gel films had a significantly-increased strength, but a decreased elongation at break. Although a breaking strain decreased after crosslinking, breaking strengths of E-Col-UV (1.33±0.19 MPa) and E-Col-GA (5.22±0.73 MPa) were significantly improved; and it was speculated that E-Col-GA had a high mechanical strength after crosslinking due to a high crosslinking density, indicating that E-Col-GA could withstand high external shearing and stretching during a surgery and a postoperative repair to maintain the mechanical stability of the material and an affected site.

Further, a mechanical test was conducted to determine suture strengths of the materials. As shown in FIG. 19C, E-Col-GA had higher suture resistance than E-Col-UV. As shown by quantification results of suture resistance of the materials in FIG. 19D, the suture resistance (1.75±0.3 N) of E-Col-GA was significantly higher than the suture resistance (0.33±0.13 N) of E-Col-UV, indicating that the suture resistance of E-Col-GA was strong enough to allow implantation with a suture line.

### Example 33: Cell Adhesion and Proliferation of HCECs on an E-Col-GA Film

With reference to Examples 30 and 32, the E-Col-GA gel film was selected for cell experiments.
(1) Cell adhesion experiment: The prepared E-Col-GA gel film was cut into round samples each with a diameter of 10 mm, then placed in a 48-well plate, soaked in 75% alcohol and irradiated with a UV light source overnight to allow sterilization, and then rinsed with PBS multiple times. HCECs were inoculated on the film at a density of 5×10⁴ cells/well, and cultivated for 12 h. Subsequently, the film was fixed with 2.5% glutaraldehyde for 20 min, then washed twice with PBS, and then gradually dehydrated with 50% ethanol, 70% ethanol, 90% ethanol, and 100% ethanol for 10 min each time. Isoamyl acetate was added dropwise, the film was dried in an oven, and the adhesion of the cells was observed by SEM (S-4800, Hitachi).
(2) Cell proliferation experiment: A cell inoculation experiment was conducted according to the same steps as above; and cells were stained with a Live/Dead cell stain at different stages and then observed by confocal laser scanning microscopy (CLSM) (Nikon AIR). After the cells were cultivated in a 37° C. incubator (5% CO₂) for 1 d, 3 d, and 5 d, the proliferation of cells was evaluated through CCK8 cell viability assay (DOJINDO), where 4 replicates were set for each group, and then an average was taken.
(3) Cell migration experiment: The prepared gel film was cut into a round sample with a diameter of 10 mm, then placed in a 48-well plate, soaked in 75% alcohol and irradiated with a UV light source overnight to allow sterilization, and then rinsed with PBS multiple times to remove the residual alcohol, where it was ensured that the material was tightly attached to a bottom of the plate. HCECs were inoculated on a surface of the film at a density of 10×10⁴ cells/well, and cultivated; and in a blank control group, cells were inoculated on a well plate at the same density. When it was observed under an inverted microscope that a surface of the material or plate was fully covered with cells, a long strip scratch was gently formed by a 200 μL pipette tip on the surface of the material or plate. At 0 h, 12 h, 24 h, and 36 h after the scratch was formed, the healing of the scratch was observed and recorded under an inverted microscope. The migration of cells at different time points was quantified by Image J. An area A₁ occupied by HCECs migrating to a scratch region was quantified (an initial scratch area was A₀), and a cell migration completion rate ρ (%) was calculated, where 4 replicates were set for each group, and then an average was taken:

$$ρ ( \% ) = ( {A}_{0} / {A}_{1} ) \times 100 \% . \qquad formula 1$$

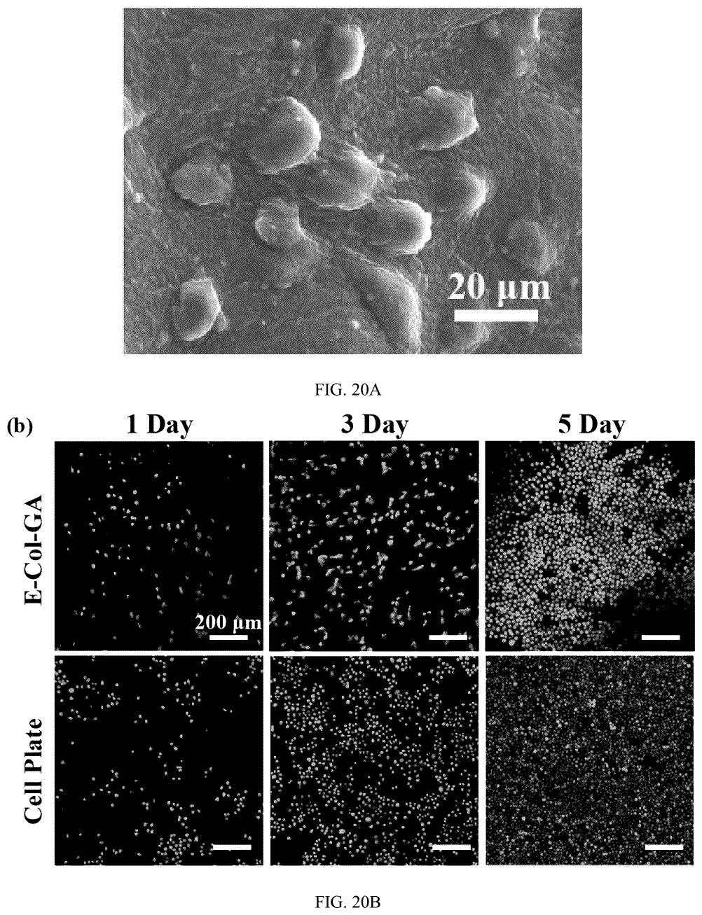

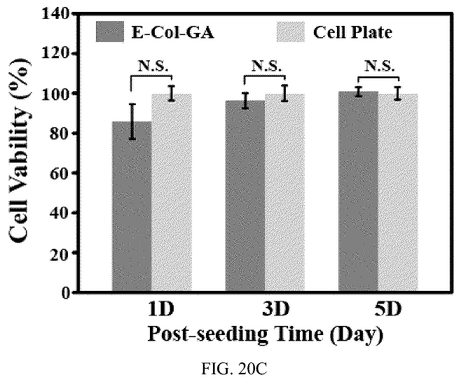

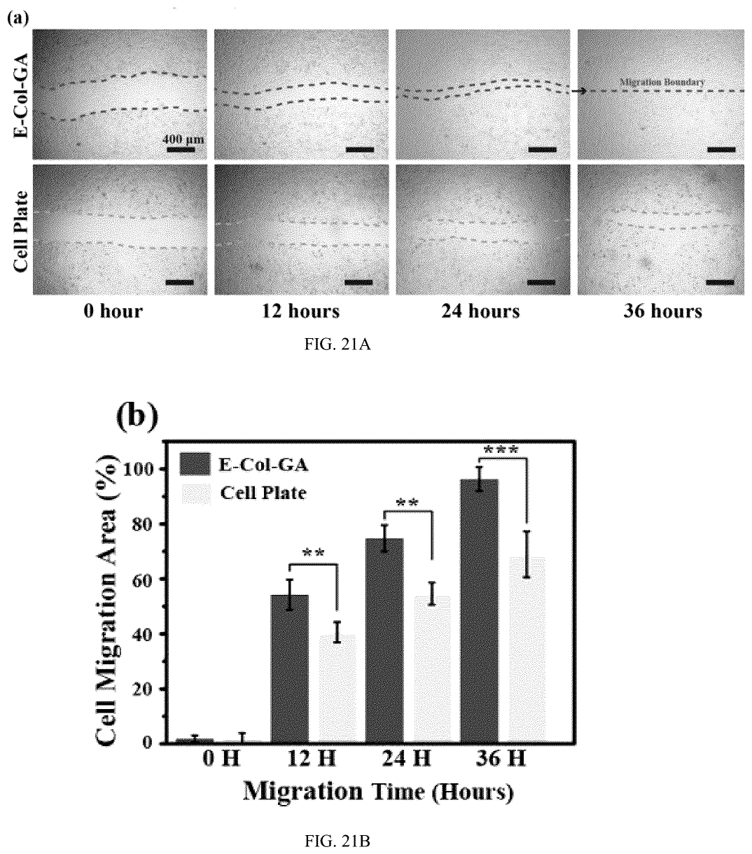

As shown in FIG. 20A, HCECs could well adhere to a surface of the E-Col-GA film in a spreading state; and HCECs inoculated on the E-Col-GA film and the tissue culture well plate underwent sustained proliferation on day 1, day 3, and day 5, and no significant dead cells were found during the 5-day observation period. As shown in FIG. 20B, the E-Col-GA film exhibited excellent cell compatibility, and this observation result was confirmed by a cellular metabolism activity quantified by CCK-8, where HCECs inoculated on the E-Col-GA film and the tissue culture well plate control exhibited high viabilities (higher than 90%) on day 1, day 3, and day 5 after inoculation. As shown in FIG. 20C, the E-Col-GA film had excellent cell compatibility and could support the adhesion and proliferation of HCECs. The in vitro scratch assay results in FIG. 21A showed that the migration of epithelial cells inoculated on a surface of the E-Col-GA gel film could be completed within less than 36 h to fill a scratch region (with a width of about 500 μm), and in contrast, epithelial cells migrated slowly at a bottom of the well plate and still failed to fill a scratch region at 36 h. To quantify the migration of cells to a scratch region, a percentage of an area occupied by cells migrating to the scratch region in an initial area of the scratch region was calculated at different time points. Results showed that, at 12 h, 24 h, and 36 h after scratching, cell migration completion rates on the E-Col-GA gel film were significantly higher than corresponding cell migration completion rates on the control (tissue culture well plate). As shown in FIG. 21B, a cell migration completion rate on the E-Col-GA gel film at 36 h basically reached 100%, which was 33% higher than a corresponding cell migration completion rate on the control (tissue culture well plate), indicating that the E-Col-GA film was conducive to the migration of HCECs.

### Example 34: A Transplantation and Repair Experiment of an E-Col-GA Film at a Corneal Lamella In Vivo

### (1) Construction of a Defective Corneal Lamella Model

10-12-week-old male New Zealand white rabbits (provided by the Shanghai Eye & ENT Hospital, an affiliate of Fudan University) were selected, and eyes of these rabbits were subjected to lamellar (thickness: 250 μm) keratectomy under sterile conditions. The rabbits were subjected to general anesthesia, and then to local anesthesia through intramuscular injection of a xylazine hydrochloride injection (1 mg/kg to 2 mg/kg) and application of oxybuprocaine hydrochloride eye drops; a negative-pressure vacuum trephine with a diameter of 7 mm was used to create a defect with a depth of about 250 μm in a central cornea; then, lamellar keratectomy was conducted with a scalpel at the same depth; and the E-Col-GA film and a commercial acellular porcine cornea matrix (denoted as commercial) were filled and sutured to different rabbit corneal defect sites, and a defect site without any material implanted was set as a blank control.

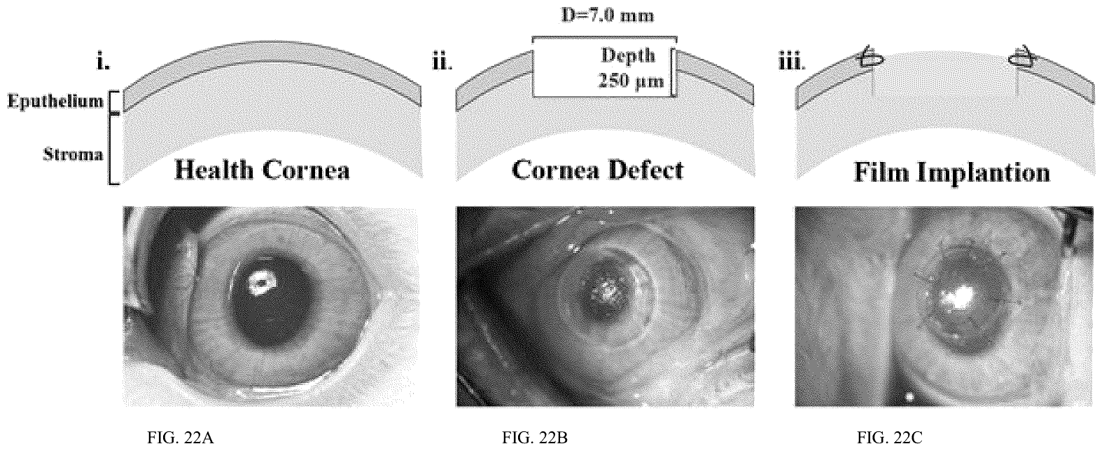

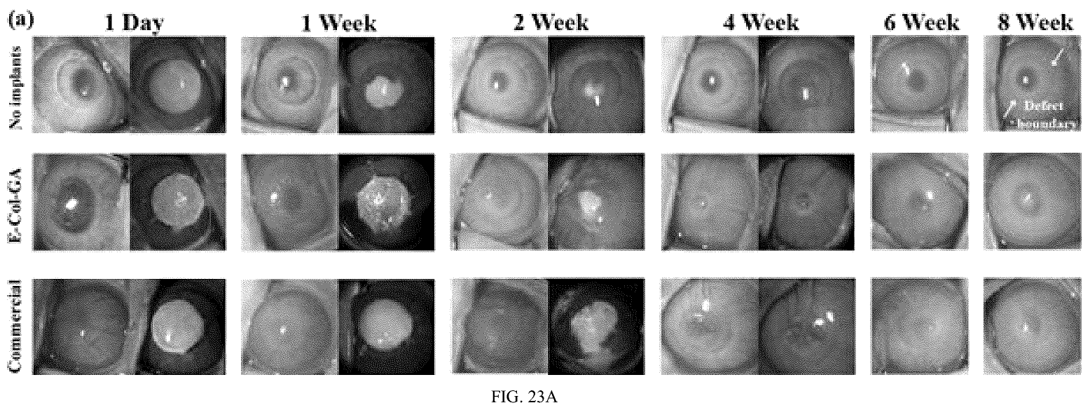

A schematic diagram of a healthy cornea, a cornea with a defect (diameter: 7 mm, and depth: 250 μm) constructed, and a cornea with E-Col-GA transplanted and actual pictures thereof during a surgery were shown in FIGS. 22A-22C. As shown in the actual pictures, E-Col-GA could be sutured to a defect site while presenting a high-transparency character.

### (2) Examination by Postoperative Slit Lamp Bioscopy

1, 2, 4, 6, and 8 weeks after the surgery, corneal tissues of the rabbits under general anesthesia were subjected to non-destructive observation under a slit lamp. Transparencies of an implanted film material and a surrounding cornea were evaluated with a slit and a broad beam at a magnification of ×16 in a white light mode. To evaluate the migration of CECs on an implanted film, a fluorescein sodium ophthalmic strip was wetted and then used to fluorescently stain a defect region at an implantation site, and a fluorescently-stained region was photographed under a cobalt-blue slit lamp microscope.

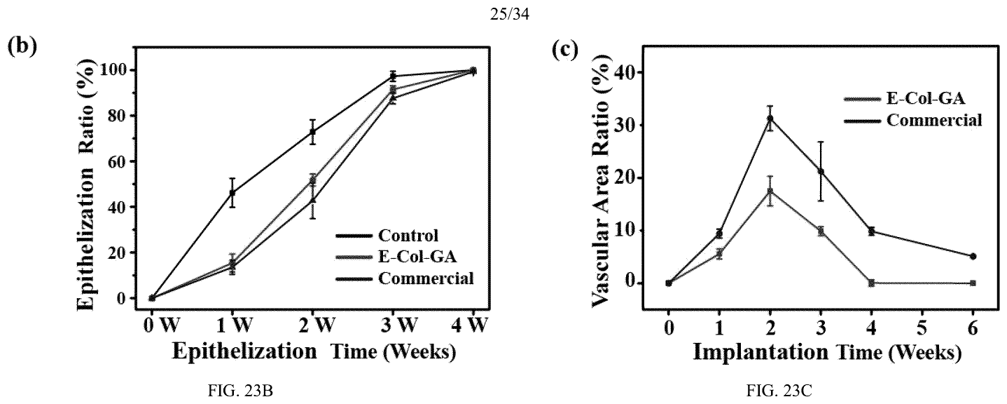

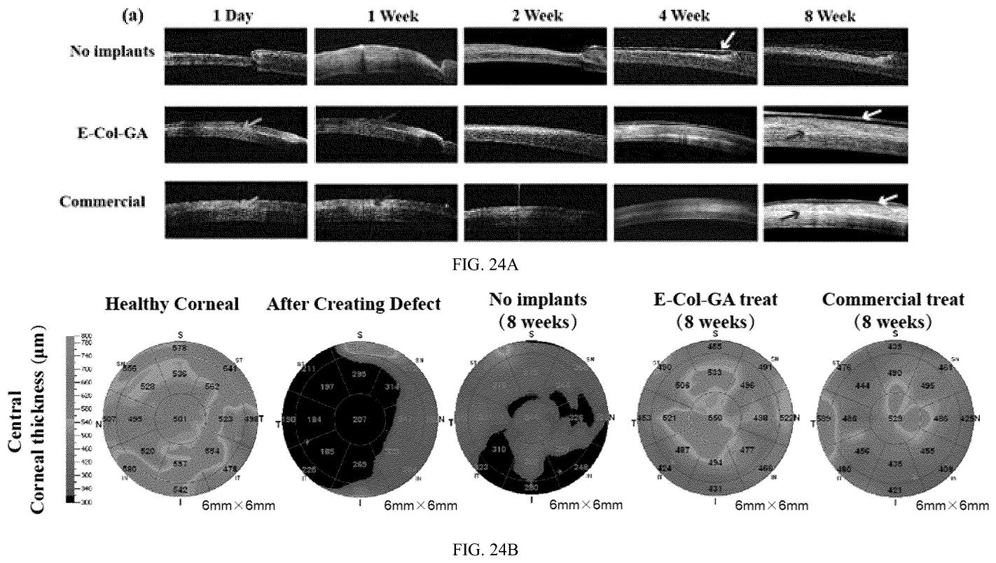

Healing conditions 8 weeks after treatments in different groups were shown in FIGS. 23A-23C. 1 week after the surgery, in an implanted material-free blank group, an obvious edema occurred to cause a specified degree of turbidity of a cornea, and the edema gradually disappeared over time to recover a transparent state, but a distinct defect boundary (marked by a white arrow) could still be observed after the 8-week observation period was completed. In contrast, in an experimental group (E-Col-GA) and a positive control group (commercial acellular porcine cornea matrix), after a material was implanted, a specified immune response first occurred, including emergence of some fine blood vessels around the material and a slight edema of a tissue, which was a normal immune response after the material was implanted to a corneal defect site; but the blood vessels generated under stimulation gradually disappeared over time. As shown in FIG. 23C, a proportion of an area of a vascularized region after implantation was quantitatively calculated by the software Image J; obvious blood vessels were generated within 2 weeks after implantation in both the experimental group and the positive control group, but the experimental group had a smaller vascularized region than the positive control group, and two weeks after implantation, blood vessels initially generated in the two groups gradually disappeared; and 6 weeks after implantation, blood vessels initially generated in the experimental group completely disappeared, while there were still some blood vessels that did not disappear in the positive control group. 8 weeks after implantation, a cornea of the experimental group basically became completely transparent, but the positive control group still had a specified degree of turbidity and was not restored to a normal transparency of a cornea, which was presumably related to a significant immune response after implantation. Compared with the blank control group, no obvious defect boundary was observed in both implanted groups after the 8-week repair period was completed.

In addition, the migration of rabbit corneal epithelial cells (RCECs) on the gel film was observed. Cobalt-blue slit lamp pictures acquired after fluorescein staining showed that sizes of corneal epithelial defects in different groups gradually decreased. As shown in FIG. 23A, there was a significant migration of epithelial cells in different groups (an epithelial defect was stained green by fluorescein), but epithelization rates were different; and in the blank control group, a migration rate of CECs was the highest because CECs grew along a surface of a defective stromal layer. The experimental group (E-Col-GA) exhibited a comparable or even higher corneal epithelization rate to or than the positive control group (acellular porcine cornea matrix), and underwent almost complete epithelization 4 weeks after the surgery, as shown in FIG. 23B.

### (3) Examination by Postoperative OCT

OCT is a high-resolution cross-sectional non-contact imaging system, and can be used to detect the fusion of a material with a tissue. OCT was conducted for the rabbits under general anesthesia 1, 2, 4, and 8 weeks after the surgery.

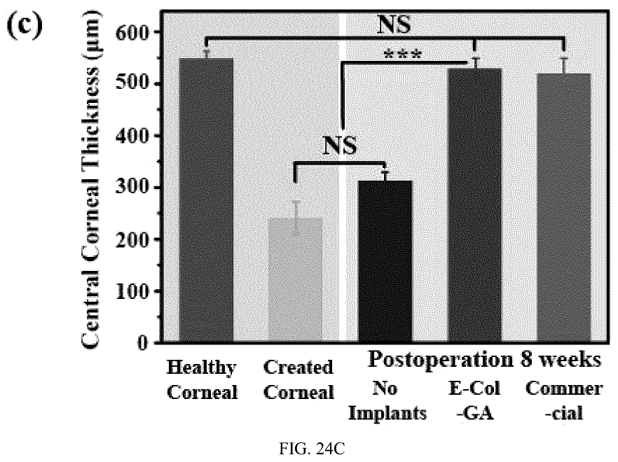

Tomographic images of defects in different groups 0 to 8 weeks after the surgery were first acquired, as shown in FIG. 24A. In the implanted material-free group, an obvious edema occurred 1 week after the surgery, and 2 weeks after the surgery, the edema basically disappeared, but an obvious defect still could be observed on a corneal stromal layer. Although complete epithelization could be observed 4 weeks after the surgery, a thickness of the corneal stromal layer at a defect basically could not return to a normal level even 8 weeks after the surgery (as shown by a white arrow in an image). In contrast, an experimental group and a positive control group both were basically completely restored to a thickness of a healthy cornea one day after the surgery. One day after the surgery, an interface between a material and a stromal layer could be observed (as shown by an orange arrow in an image), and 1 week after the surgery, the interface between the material and the stromal layer had gradually blurred (as shown by a red arrow in an image), indicating the fusion of the material with the autologous stromal tissue. The gradual epithelization could be observed during the fusion of the material with the autologous stromal tissue, and the generation of a complete epithelialized tissue could be observed 8 weeks after implantation (as shown by a white arrow in an image); and a region indicated by a red arrow also showed the basic fusion of the material with the autologous stromal layer.

Further, in a full corneal thickness observation mode of OCT, a full corneal thickness topographic map 8 weeks after the surgery was acquired, and a thickness was quantitatively determined, as shown in FIGS. 24B-24C. A healthy cornea of a rabbit had a thickness of about 550 μm. After a defect was constructed, a thickness of a cornea decreased significantly, and a measured thickness of a defective cornea was about 200 μm. 8 weeks after the surgery, a thickness of a cornea in the implanted material-free group increased to some extent. In contrast, in the experimental group and the positive control group, the thickness of the healthy cornea was basically restored 8 weeks after implantation.

### Example 35: Mechanical Strengthening for an E-Col Film Based on a Hofmeister Effect

E-Col was prepared by the same method as in Example 1, where gel E-Col with a thickness of about 500 μm was obtained by controlling a current intensity and an application time of the current intensity. For comparison, a collagen solution obtained after dialysis was also used to prepare a collagen film S-Col by a solution method, including: a pH of the acidic collagen solution (5 mg/mL; and pH=3.5) was adjusted with 0.5 M NaOH to 7.2, then a resulting collagen solution was poured into a round film Petri dish (a collagen content per unit area was the same as a mass of EDP-assembled collagen per unit area), and the Petri dish was incubated at 37° C. for 12 h to allow complete gelation; and a resulting gel was dehydrated for 48 h at room temperature to produce a milky-white translucent gel film S-Col with a thickness of about 500 μm.

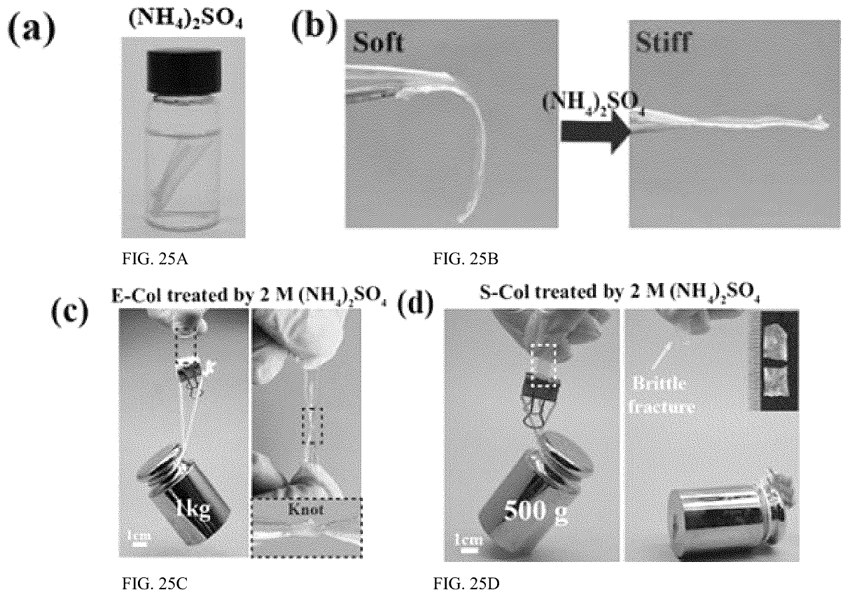

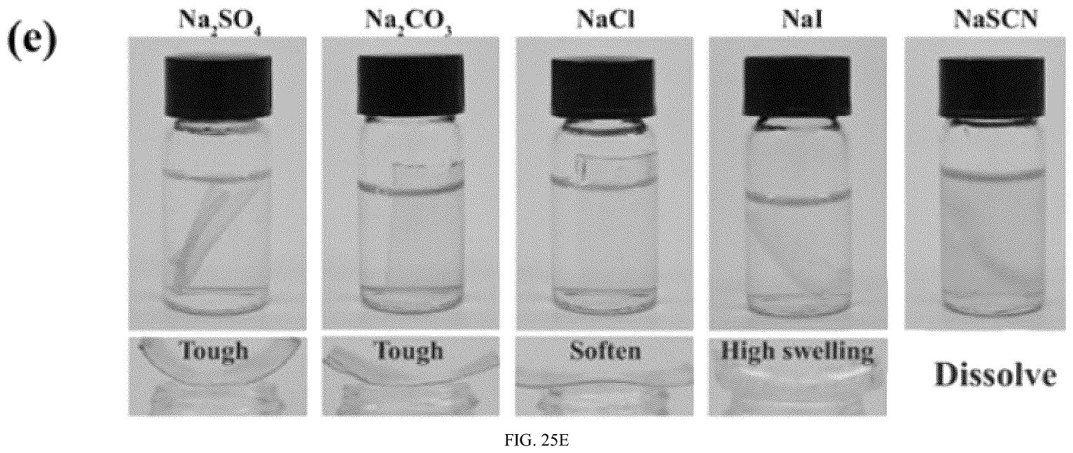

The prepared E-Col and S-Col films each were cut into rectangular sample strips each with a length of 30 mm and a width of 10 mm, and then soaked in a (NH₄)₂SO₄ (2 mol/L) solution for 24 h (as shown in FIG. 25A), and changes in mechanical properties of the films were observed. As shown in FIG. 25B, a network of the E-Col collagen film could undergo a significant stiffening phenomenon under classical stimulation of Hofmeister salt-ammonium sulfate, which significantly strengthened the mechanical properties of the collagen film.

After a mechanical strengthening effect of (NH₄)₂SO₄ for the E-Col collagen film was investigated, with the S-Col gel film assembled in a solution as a control, differences among (NH₄)₂SO₄-induced Hofmeister mechanical strengthening effects for different assembled structures were investigated. As shown in FIGS. 25C-25D, after an E-Col network was treated with (NH₄)₂SO₄ (2 M, 24 h), a resulting transparent E-Col gel film could withstand a load of 1 kg while retaining the flexibility of the network, and the film could be knotted without rupturing. In contrast, after an S-Col network was treated by the same method, a small strengthening effect was allowed, and a treated film could not withstand a load of 500 g and was prone to brittle fracture.

In order to investigate the impacts of different Hofmeister ions on the mechanical properties of E-Col, the following five different salts were selected to soak E-Col: CO₃²⁻, SO₄²⁻, Cl⁻, SCN⁻, and I⁻. As shown in FIG. 25E, a mechanical strength of the gel film soaked in a CO₃²⁻ or SO₄²⁻ solution significantly increased; the gel film soaked in a NaI solution swelled; and the gel film was directly dissolved in a NaSCN solution.

### Example 36: Changes in a Water Content of an E-Col Film Treated Under Different Hofmeister Salt Concentrations (Dehydration Effects)

The collagen films (the E-Col film and the S-Col film) prepared in Example 35 were adopted. The collagen films each were soaked in a series of (NH₄)₂SO₄ solutions with different concentrations (0 M, 1 M, 2 M, 2.5 M, and 4 M) at room temperature for 24 h, and then water contents in the collagen films after being soaked were tested.

As shown in the table below, the water contents in the two collagen films both decreased to some extent after being soaked, and decreased significantly with the increase of a (NH₄)₂SO₄ concentration. There was no significant difference between the water contents in the two collagen films after being soaked.

Comparison of Water Contents of the Collagen Films Treated Under Different (NH₄)₂SO₄ Concentrations

| (NH₄)₂SO₄ | E-Col Film | S-Col Film |
|---|---|---|
| Concentration [M] | [%] | [%] |
| 0 | 89.68 ± 2.14 | 88.69 ± 1.03 |
| 1 | 76.47 ± 1.94 | 79.04 ± 3.02 |
| 2 | 56.37 ± 1.86 | 57.58 ± 4.65 |
| 2.5 | 48.93 ± 3.47 | 50.76 ± 2.69 |
| 4 | 38.77 ± 2.85 | 46.69 ± 1.03 |

### Example 37: Quantitative Characterization of Mechanical Properties of an E-Col Film Strengthened Based on a Hofmeister Effect

E-Col and S-Col collagen films were prepared by the same methods as in Example 35. The collagen films (10 mm×0.5 mm×30 mm) each were soaked in ammonium sulfate solutions with different concentrations (1 M, 2 M, 2.5 M, and 4 M) at room temperature for 12 h to strengthen the hydrophobic and H bonding interactions, and then an Electro-Force 3200 biodynamic tester was used to investigate the mechanical properties of the collagen films at room temperature. A sample was stretched by a fixture at a strain rate of 10 mm/min, a Young's modulus (MPa) of the sample was calculated based on a slope factor of an initial linear region of a stress-strain curve, and a toughness value of the sample was calculated based on an integral area of a tensile stress-strain curve (MJ/m³).

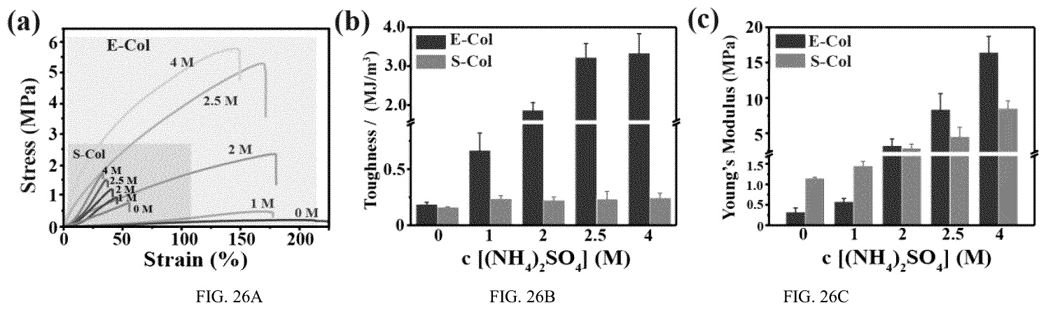

As shown by qualitative stress-strain curves in FIG. 26A, a mechanical property strengthening effect of the (NH₄)₂SO₄ treatment for the E-Col gel film was significantly dependent on a concentration of (NH₄)₂SO₄, and the (NH₄)₂SO₄ treatment had a much smaller strengthening effect for an S-Col gel film than for the E-Col gel film. It was summarized in FIG. 26B that, after S-Col and E-Col networks both were strengthened by the 4 M (NH₄)₂SO₄ treatment, a Young's modulus of the E-Col film increased by 50 times and a Young's modulus of the S-Col film increased merely by 6 times. It was summarized in FIG. 26C that the 4 M (NH₄)₂SO₄ treatment increased the toughness of the E-Col film by 16 times, but this treatment made basically no contribution to the toughness strengthening for the S-Col film. The above results showed that, after the (NH₄)₂SO₄ treatment, the toughness of the E-Col network was significantly improved compared with the toughness of the S-Col network, indicating that the collagen gel films with different assembled structures had different mechanical responses for a Hofmeister effect.

### Example 38: Quantitative Characterization of Mechanical Properties of an E-Col Film Strengthened Based on a Hofmeister Effect

An E-Col gel film was prepared by the same method as in Example 35. The E-Col gel film (10 mm×0.5 mm×30 mm) was soaked in sodium carbonate (Na₂CO₃) solutions with different concentrations (1 M, 2 M, and 2.5 M) at room temperature for 12 h, and then an Electro-Force 3200 biodynamic tester was used to investigate the tensile properties of the gel film at room temperature.

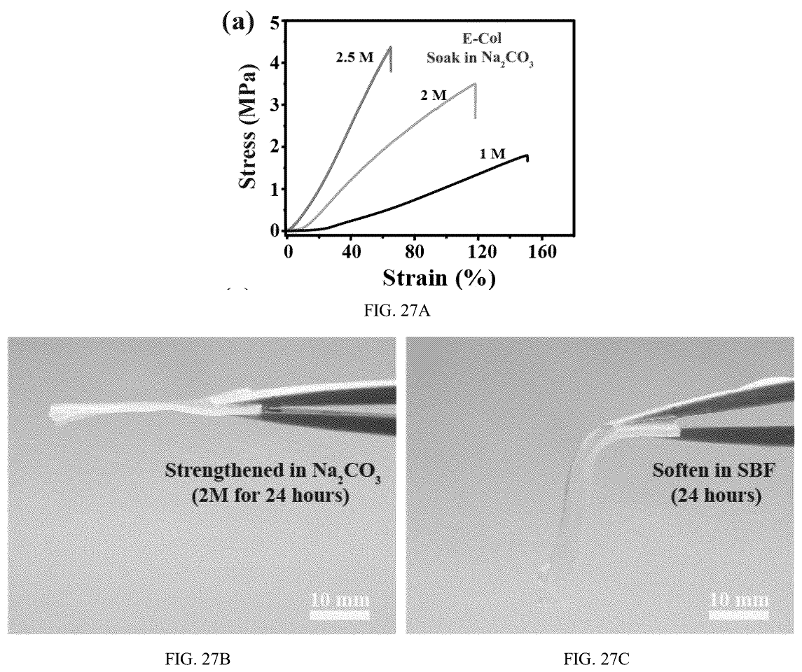

A stretching rate was set at 10 mm/min to obtain a stress-strain curve of the gel film. As shown by stress-strain curves of the E-Col gel film treated with Na₂CO; of different concentrations in FIG. 27A, Na₂CO; as a Hofmeister salt with a strong hydration ability could also strengthen the E-Col network, and a strengthening effect increased with the increase of a concentration of the salt.

An E-Col gel film was prepared by the same method as in Example 2. The E-Col gel film (10 mm×0.5 mm×30 mm) was soaked in a 2 M Na₂CO₃ solution at room temperature for 12 h. As shown in FIGS. 27B-27C, the E-Col gel film was significantly strengthened after being treated with 2 M Na₂CO₃ for 24 h, but the strengthened E-Col gel film was gradually softened to an initial soft state after being treated in SBF (Guangzhou Yazhi Biotechnology Co., Ltd., PH1820) for 24 h, that is, a strengthening process of the E-Col network by Na₂CO; was a reversible process, and the strengthened E-Col gel film returned to a soft state after Hofmeister salt ions were leached out, indicating that a mechanical strengthening effect based on a Hofmeister effect for the E-Col film was reversible.

### Example 39: Use of a Mechanically-Strengthened E-Col Film for In Vivo Artery Banding

### (1) Construction of a PAB Experimental Model

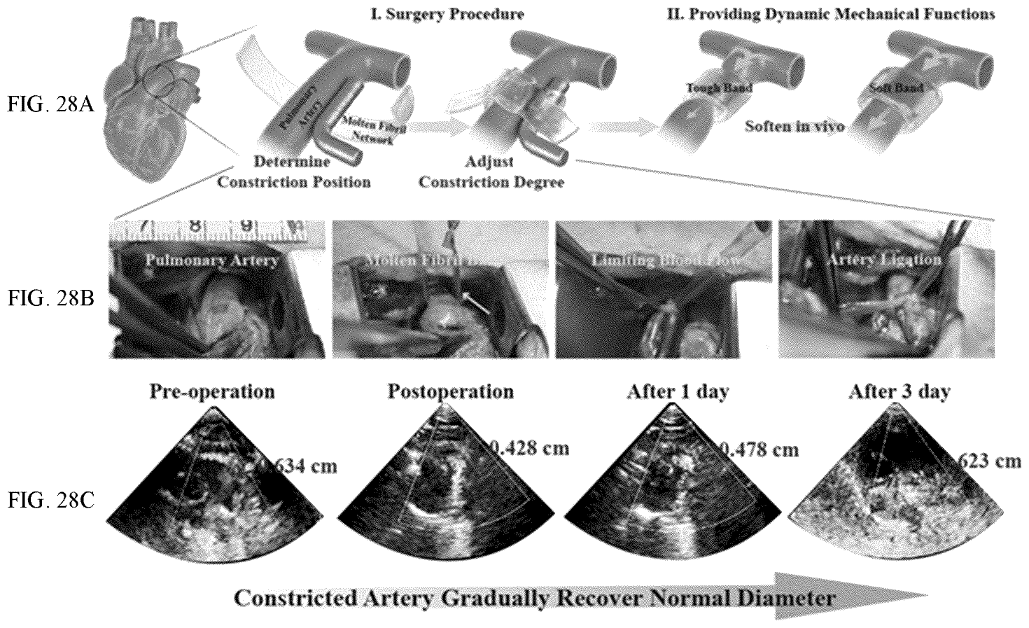

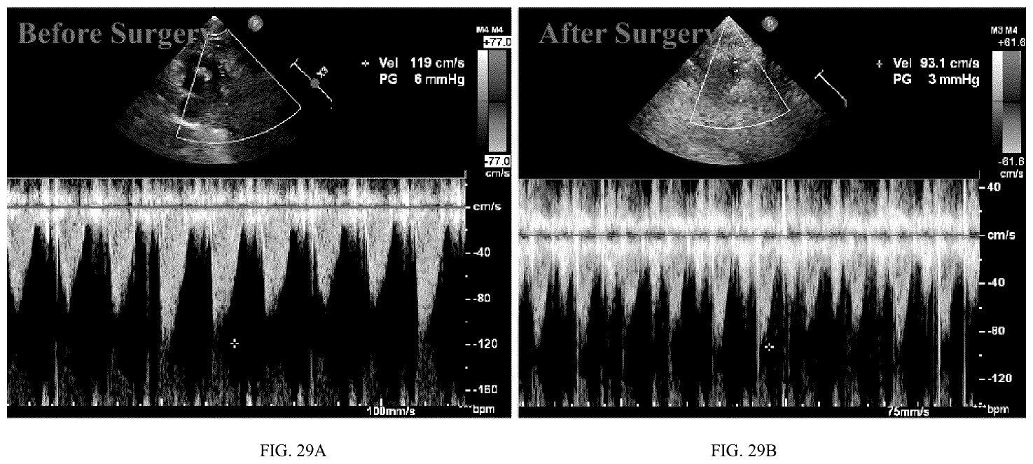

2-month-old New Zealand white rabbits were selected and anesthetized through ear vein injection of pentobarbital at 40 mg kg⁻¹ to 50 mg kg⁻¹. Before PAB, color Doppler was first used to observe a pulmonary artery and record a diameter of the pulmonary artery, and then a VEL and a PG of each rabbit were measured. Preparation works such as endotracheal intubation, breathing support, inhalation anesthesia, and nutrient solution supply were completed in advance. The left ventricle (LV) was exposed after thoracotomy. A pulmonary artery was first exposed, and a position for artery banding was determined; a collagen microfiber hydrogel band prepared by EDP (which was strengthened with 2 M Na₂CO₃) was wrapped around the pulmonary artery, and a sliding hydrogel knot was made; and then a degree of the artery banding was adjusted, the sliding hydrogel knot was tightened, and an excess part of the hydrogel band was removed. After the surgery was completed, a rabbit chest was closed according to conventional clinical steps. After the surgery, reduced diameter, VEL, and PG values of the pulmonary artery were tested to determine a pressure reduction effect. On day 1 and day 3 after the surgery, color Doppler was used to observe the recovery of the diameter of the pulmonary artery, as shown in FIGS. 28A-28C.

### (2) Postoperative Color Doppler Image Results

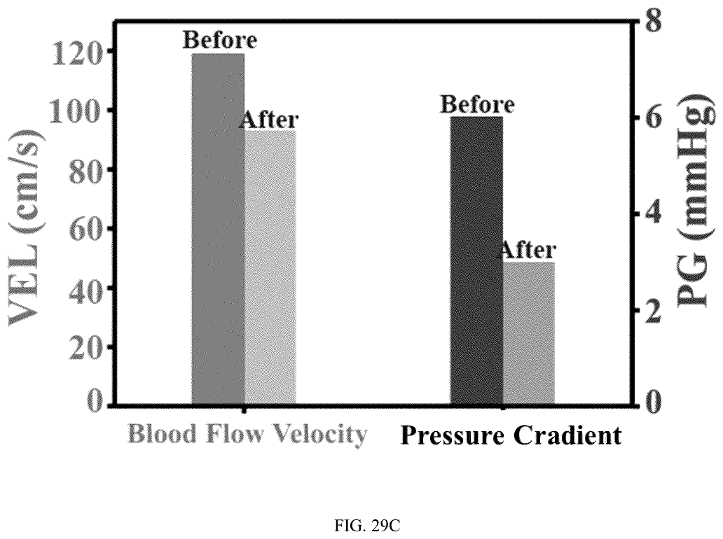

Experimental New Zealand rabbits were adopted as an animal model, and a diameter of a pulmonary artery was reduced with the E-Col film strengthened by Na₂CO₃ as a surgical band, as shown in FIG. 28B. As shown by color Doppler images of a heart in FIG. 28C, the surgical band reduced the diameter of the pulmonary artery from a preoperative diameter Φ=0.63 cm to a postoperative diameter Φ₀=0.43 cm, with a reduction of about 68%, indicating that E-Col strengthened by Na₂CO₃ could provide a high mechanical strength to significantly reduce a diameter of a pulmonary artery; and the diameter of the pulmonary artery returned to 75% of the preoperative diameter of the pulmonary artery (Φ₁=0.48 cm) on day 1 after the surgery, and returned to the preoperative diameter of the pulmonary artery on day 3 after the surgery. VELs and PGs of artery vessels before and after the surgery that were tested by Doppler ultrasonography were shown in FIGS. 29A-29C. A VEL decreased from 119 cm/s before the surgery to 93.1 cm/s after the surgery, and a PG significantly decreased from 6 mmHg before the surgery to 3 mmHg after the surgery, indicating that a strengthened E-Col band implanted around the pulmonary artery could significantly constrict the pulmonary artery to allow the short-term restriction of VEL and the reduction of a blood flow pressure.

The above examples are merely few examples of the present application, and do not limit the present application in any form. Although the present application is disclosed as above with preferred examples, the present application is not limited thereto. Some changes or modifications made by any technical personnel familiar with the profession using the technical content disclosed above without departing from the scope of the technical solutions of the present application are equivalent to equivalent implementation cases and fall within the scope of the technical solutions.

What is claimed is:

1. A preparation method of a collagen material stripped from electrodes, comprising the following step:
subjecting a collagen solution to an electro-deposition (EDP) to obtain the collagen material on the electrode, wherein the collagen solution comprises hydrogen peroxide and/or acetic acid.

2. The preparation method according to claim 1, wherein a pH of the collagen solution is 1.5 to 4.0.

3. The preparation method according to claim 1, wherein a concentration of the collagen solution is 1 mg/mL to 20 mg/mL.

4. The preparation method according to claim 1, wherein a volume percentage of the hydrogen peroxide in the collagen solution is 5% to 17%.

5. The preparation method according to claim 1, wherein the EDP is conducted under the following conditions:
temperature: 0° C. to 30° C.; and time: 8 min to 60 min.

6. The preparation method according to claim 1, wherein during the EDP, a current density is 0.5 mA/cm² to 10 mA/cm², and a voltage is 0.22 V/cm² to 1.67 V/cm².

7. The preparation method according to claim 1, wherein during the EDP, a distance between the electrodes is 1.0 cm to 2.5 cm.

8. The preparation method according to claim 1, wherein a cathode of the electrodes is one selected from a stainless steel, a carbon paper, a carbon cloth, a Pt electrode, a gold electrode, a graphite electrode, and a Ti electrode.

9. The preparation method according to claim 1, wherein an anode of the electrodes is one selected from a stainless steel, a carbon paper, a carbon cloth, a Pt electrode, a gold electrode, and a graphite electrode.

10. The preparation method according to claim 1, comprising the following steps:
S1, mixing a collagen-containing solution and the acetic acid to obtain an intermediate collagen solution;
S2, adding a hydrogen peroxide-containing mixture to the intermediate collagen solution to obtain the collagen solution; and
S3, placing the collagen solution in an electrolytic cell, and conducting the EDP to obtain the collagen material.

11. The preparation method according to claim 1, wherein an arrangement manner of the electrodes comprises: vertically arranging two electrodes in parallel in an electrolytic cell, or horizontally arranging the two electrodes in parallel in the electrolytic cell.

12. A collagen film, wherein a preparation method of the collagen film comprises:
dissolving a mixture comprising a collagen material in a solvent, and recycling to obtain the collagen film,
wherein the collagen material is prepared by the preparation method according to claim 1.

13. The collagen film according to claim 12, wherein the collagen film has a thickness of 180 μm to 550 μm.

14. The collagen film according to claim 12, wherein the collagen film has a uniform appearance and is highly-transparent in dry and wet states.

15. The collagen film according to claim 12, wherein the collagen film is obtained by linking short range-oriented collagen microfibers through non-covalent bonds.

16. The collagen film according to claim 12, wherein the collagen film has a dense collagen arrangement.

17. A method of preparing a collagen film with highly-oriented and crystalline collagen fibers using a collagen material, comprising the following steps:
A1, stretching the collagen material in a length direction of the collagen material to produce a first collagen material;
A2, incubating a mixture comprising the first collagen material and a phosphate-buffered solution (PBS) to obtain large-diameter collagen fibers; and
A3, subjecting the large-diameter collagen fibers to chemical crosslinking to obtain the collagen film with the highly-oriented and crystalline collagen fibers,
wherein the collagen material is prepared by the preparation method according to claim 1.

18. The method according to claim 17, wherein in step A1, a strain degree of the stretching is Ts, and 50%≤Ts≤200%.

19. The method according to claim 17, wherein in step A2, a concentration of the PBS is 0.05 M to 0.5 M.

20. The method according to claim 17, wherein in step A2, the incubating is conducted for 6 h to 72 h.

21. The method according to claim 17, wherein in step A3, the chemical crosslinking comprises photocrosslinking, glutaraldehyde crosslinking, genipin crosslinking, and polyphenol crosslinking.

22. The method according to claim 21, wherein the photocrosslinking is conducted as follows:
soaking the large-diameter collagen fibers in a 0.2 mg/mL to 3.0 mg/mL riboflavin solution, and allowing the photocrosslinking under an ultraviolet (UV) irradiation for 1 d to 3 d.

23. The method according to claim 21, wherein the glutaraldehyde crosslinking is conducted as follows:
soaking the large-diameter collagen fibers in a 0.1% to 1% glutaraldehyde solution, and allowing the glutaraldehyde crosslinking for 10 min to 2 h.

24. The method according to claim 21, wherein the genipin crosslinking is conducted as follows:
soaking the large-diameter collagen fibers in a genipin solution with a mass percentage of 0.2% to 2.0%, and allowing the genipin crosslinking for 8 h to 14 h.

25. The method according to claim 21, wherein the polyphenol crosslinking is conducted as follows:
soaking the large-diameter collagen fibers in an aqueous solution of proanthocyanidin (PAC), tannic acid (TA), or gallic acid (GA) with a mass percentage of 0.1% to 2.0%, and allowing the polyphenol crosslinking for 8 h to 14 h.

26. The method according to claim 17, wherein in step A1, a stretched collagen material is soaked in ethanol to temporarily fix an oriented structure of the stretched collagen material.

27. The method according to claim 17, wherein in step A2, two ends of the first collagen material are fixed during an ion incubation to make the first collagen material undergo a continuous external action force without shrinking.

28. The method according to claim 17, wherein the collagen film comprises long range-orientated collagen fibers with distinctive D-band characteristics.

29. The method according to claim 17, wherein the collagen film has a Young's modulus close to a Young's modulus of a native tendon.

30. A method of using the collagen film obtained in the method according to claim 17 in an artificial tendon.

31. A method of preparing an artificial cornea using a collagen solution, comprising the following steps:
B1, acquiring a cathode with a curvature range of 7.8 to 8.5 as a working electrode of an electrolytic cell;
B2, placing a mixture comprising the collagen solution in the electrolytic cell, and conducting the EDP to obtain the collagen material on the cathode; and
B3, subjecting the collagen material to chemical crosslinking to obtain the artificial cornea,
wherein the collagen solution is selected from the collagen solution in the preparation method according to claim 1.

32. The method according to claim 31, wherein the EDP in step B2 is conducted under the following conditions:
temperature: 0° C. to 30° C.; and time: 8 min.

33. The method according to claim 31, wherein the chemical crosslinking in step B3 comprises photocrosslinking, glutaraldehyde crosslinking, genipin crosslinking, and polyphenol crosslinking;
the photocrosslinking is conducted as follows:
soaking the collagen material in a 0.2 mg/mL to 3.0 mg/mL riboflavin solution, and allowing the photocrosslinking under an UV irradiation for 1 d to 3 d;
the glutaraldehyde crosslinking is conducted as follows:
soaking the collagen material in a 0.1% to 1% glutaraldehyde solution, and allowing the glutaraldehyde crosslinking for 10 min to 2 h;
the genipin crosslinking is conducted as follows:
soaking the collagen material in a genipin solution with a mass percentage of 0.2% to 2.0%, and allowing the genipin crosslinking for 8 h to 14 h; and
the polyphenol crosslinking is conducted as follows:
soaking the collagen material in an aqueous solution of proanthocyanidin (PAC), tannic acid (TA), or gallic acid (GA) with a mass percentage of 0.1% to 2.0%, and allowing the polyphenol crosslinking for 8 h to 14 h.

34. An artificial cornea, wherein the artificial cornea is matched with a corneal curvature; and
the artificial cornea is obtained in the method according to claim 31.

35. A method of using an artificial cornea in a cornea repair, comprising the following steps:
D1, cutting the artificial cornea;
D2, filling and suturing a cut artificial cornea to a corneal defect site; and
D3, allowing the cornea repair for at least 2 weeks,
wherein the artificial cornea is obtained in the method according to claim 31.

36. A method of preparing a bandage using a collagen material, comprising the following step:
placing a mixture comprising the collagen material in a salt solution to obtain the bandage,
wherein the collagen material is prepared by the preparation method according to claim 1.

37. The method according to claim 36, wherein the salt solution is a solution of a soluble salt of a Hofmeister ion.

38. The method according to claim 37, wherein the Hofmeister ion is selected from at least one of CO₃²⁻, SO₄²⁻, S₂O₃²⁻, H₂PO₄⁻, NO₃⁻, CH₃COO⁻, ClO₄⁻, F⁻, Cl⁻, and Br⁻.

39. The method according to claim 36, wherein a concentration of the salt solution is 0.1 M to 4 M.

40. The method according to claim 36, wherein the mixture is placed in the salt solution for 0.5 h to 60 h.

41. A bandage comprising a short range-oriented collagen film,
wherein the bandage is obtained in the method according to claim 36.

42. The bandage according to claim 41, wherein the short range-oriented collagen film is allowed to capable of automatically return to a soft state and dynamically relax.

43. The bandage according to claim 41, wherein the short range-oriented collagen film has a breaking strength of 2.0 MPa to 8.0 MPa.

44. The bandage according to claim 41, wherein the short range-oriented collagen film has a Young's modulus of 9.0 MPa to 18.0 MPa.

45. The bandage according to claim 41, wherein the short range-oriented collagen film has a toughness value of 0.5 MJ/M³ to 5.5 MJ/M³.

46. The bandage according to claim 41, wherein the bandage has a thickness of 50 μm to 1,000 μm.

47. A method of using the bandage according to claim 41 in artery banding.

48. The method according to claim 47, comprising the following steps:
E1, determining a position for the artery banding;
E2, wrapping the bandage around the position for the artery banding, and making a sliding hydrogel knot; and
E3, adjusting a degree of the artery banding, and removing an excess part of the bandage.
